(19) United States
(12) Patent Application Publication
(10) Pub. No.: US 2023/0365939 A1
(43) Pub. Date: Nov. 16, 2023

(54) RAFT CULTURES AND METHODS OF MAKING THEREOF

(71) Applicant: CHILDREN'S HOSPITAL MEDICAL CENTER, Cincinnati, OH (US)

(72) Inventors: Vered Shacham-Silverberg, Sharonville, OH (US); James Macormack Wells, Cincinnati, OH (US)

(21) Appl. No.: 18/027,900

(22) PCT Filed: Sep. 22, 2021
(86) PCT No.: PCT/US21/51556

§ 371 Date: Mar. 22, 2023

Related U.S. Application Data
(60) Provisional application No. 63/083,460, filed on Sep. 25, 2020.

(51) Int. Cl. C12N 5/071

(52) U.S. Cl. CPC C12N 5/0679; C12N 2533/90; C12N 2501/119; C12N 2501/155

(57) ABSTRACT

Disclosed herein are esophageal raft culture compositions that more closely resemble native organ structures. These esophageal raft cultures may also be innervated by combination with enteric neural crest cells. These raft culture compositions are advantageous for purposes such as studying organellar function, development, and organization. Also disclosed herein are methods of producing said esophageal raft culture compositions.

### CROSS-REFERENCE TO RELATED APPLICATIONS

This application claims priority to and benefit of U.S. Provisional Patent Application No. 63/083,460, filed Sep. 25, 2020, and PCT Application No. PCT/US2021/051556, filed Sep. 22, 2021.

### STATEMENT REGARDING FEDERALLY SPONSORED R&D

This invention was made with government support under P01 HD093363 awarded by the National Institutes of Health. The government has certain rights to the invention.

### FIELD OF THE INVENTION

Aspects of the present disclosure relate generally to esophageal raft culture compositions and methods of making said esophageal raft culture compositions. The raft cultures disclosed herein more closely resemble native organ structures.

### BACKGROUND

Three-dimensional (3D) cell cultures, such as organoids, have great promise as models for biological function and development, more so compared to traditional two-dimensional culture systems. These 3D cultures have the potential to more accurately reflect characteristics of organs found in vivo for applications such as pharmacological behavior, cell signaling, cancer formation and migration, or transplant/grafting. However, in vitro formation of organ tissues that mimic the complex structures found in living organisms is still a nascent field.

### SUMMARY

There is a present need for more accurate 3D organ models and methods of making thereof that, for example, are more efficient, inexpensive, and less time consuming. There is also a present need for culture preparations that optionally avoid xenogeneic components, which may have significant safety and regulatory implications. Disclosed herein are esophageal raft cultures from differentiated anterior foregut cells and intermediate cell compositions thereof. In some embodiments, the intermediate cell compositions comprise dorsal anterior foregut cells and/or esophageal progenitor cells. Also disclosed herein are methods of producing said esophageal raft cultures and intermediate cell compositions thereof. In some embodiments, the methods comprise contacting anterior foregut cells with one or more (e.g. at least 1, 2, 3, 4) of an EGF pathway activator, a BMP pathway inhibitor, an FGF pathway activator, or a growth supplement, or any combination thereof, to differentiate the anterior foregut cells into dorsal anterior foregut cells. In some embodiments, the methods comprise contacting anterior foregut cells with one or more (e.g. at least 1, 2, 3, 4) of an EGF pathway activator, a BMP pathway inhibitor, or an FGF pathway activator, optionally a neural progenitor inhibitor, or any combination thereof, to differentiate the anterior foregut cells into dorsal anterior foregut cells. In some embodiments, the dorsal anterior foregut cells are then dissociated into single cells and cultured in a first tissue culture container to expand the dorsal anterior foregut cells and differentiate them to esophageal progenitor cells. In some embodiments, the expanded esophageal progenitor cells are then dissociated into single cells and cultured in, and/or on a surface or, an insert member (e.g., a transwell or cell insert), where the insert member is positioned within a second tissue culture container and the insert member comprises a surface that is permeable to a growth medium but not cells. In some embodiments, the insert member and second tissue culture container each comprise an amount of growth medium such that the esophageal progenitor cells are fully submerged in the growth medium. In some embodiments, the esophageal progenitor cells are then cultured in the insert member where the second tissue culture container and/or insert member contains an amount of growth medium such that the esophageal progenitor cells are only partially submerged in the growth medium to produce the esophageal raft culture. In some embodiments, the second tissue culture container is the same as the first tissue culture container. In some embodiments, the partially submerged esophageal progenitor cells or esophageal raft culture are cultured in an air-liquid interface. In some embodiments, the anterior foregut cells are differentiated from definitive endoderm cells. In some embodiments, the anterior foregut cells or definitive endoderm cells are differentiated from induced pluripotent stem cells. In some embodiments, the induced pluripotent stem cells are human induced pluripotent stem cells.

In the methods and compositions disclosed herein, the esophageal progenitor cells can also be mixed with enteric neural crest cells to prepare innervated esophageal raft cultures.

Embodiments of the present disclosure provided herein are described by way of the following numbered alternatives:

1. An in vitro esophageal raft culture comprising:
a stratified squamous epithelium layer comprising a suprabasal layer and a basal layer; and
a mesenchyme layer comprising muscle fibers;
wherein the stratified squamous epithelium is E-cadherin⁺, the suprabasal layer is KRT13⁺ and KRT8⁺, and the basal layer is SOX2⁺, P63⁺, and KRT5⁺; and
wherein the mesenchyme layer is FOXF1⁺, NKX6-1⁺, and vimentin⁺, and the muscle fibers are desmin⁺.

2. The esophageal raft culture of any one of the preceding alternatives, wherein the esophageal raft culture lacks a lamina propria layer, or has a reduced lamina propria layer compared to esophageal tissue from an adult animal of the same species as the raft culture.

3. The esophageal raft culture of any one of the preceding alternatives, further comprising a growth medium, such as DMEM/F12.

4. The esophageal raft culture of any one of the preceding alternatives, further comprising a tissue culture container and an insert member, wherein the esophageal raft culture is positioned within the insert member, and the insert member is positioned within the tissue culture container; and wherein the insert member comprises a surface that is permeable to the growth medium but not cells.

5. The esophageal raft culture of alternative 4, wherein the insert member is coated with an extracellular matrix or a component thereof.

6. The esophageal raft culture of alternative 5, wherein the extracellular matrix or a component thereof is derived from human.

7. The esophageal raft culture of alternative 5 or 6, wherein the extracellular matrix or a component thereof comprises human collagen type IV.

8. The esophageal raft culture of any one of alternatives 5-7, wherein the extracellular matrix or a component thereof does not comprise rat collage type I matrix or Matrigel.

9. The esophageal raft culture of any one of alternatives 4-8, wherein the insert member and tissue culture container each contain an amount of growth medium such that the esophageal raft culture is fully submerged in the growth medium.

10. The esophageal raft culture of alternative 9, wherein the insert member further comprises an EGF pathway activator, a ROCK inhibitor, a SMAD inhibitor, or any combination thereof, and the tissue culture container comprises an EGF pathway activator.

11. The esophageal raft culture of any one of alternatives 4-8, wherein the insert member does not contain growth medium and the tissue culture container contains an amount of growth medium such that the esophageal raft culture is partially submerged in the growth medium, wherein the stratified squamous epithelium is partially submerged or not submerged in the growth medium and forms an air-liquid interface.

12. The esophageal raft culture of alternative 11, wherein the tissue culture container comprises an EGF pathway activator.

13. The esophageal raft culture of any one alternatives 4-12, wherein the insert member comprises a pore size that is, is about, is at least, is at least about, is not more than, or is not more than about, 0.1, 0.5, 1, 2, 3, 4, 5, 6, 7, 8, 9, or 10 μm, or any pore size within a range defined by any two of the aforementioned sizes.

14. The esophageal raft culture of any one of alternatives 4-13, wherein the insert member comprises a pore size of 3 μm.

15. An in vitro cell culture comprising:
a population of esophageal progenitor cells derived from dorsal anterior foregut cells which have been treated with an EGF pathway activator, a BMP pathway inhibitor, an FGF pathway activator, or a growth supplement (e.g. CultureOne supplement), or any combination thereof.

16. The cell culture of alternative 15, further comprising a growth medium, such as Keratinocyte SFM or other serum free medium.

17. The cell culture of alternative 16, wherein the growth medium comprises an EGF pathway activator or bovine pituitary extract (BPE), or both.

18. The cell culture of alternative 17, wherein:
the EGF pathway activator is at a concentration of about 1, 2, 3, 4, 5, 6, 7, 8, 9, 10, 11, 12, 13, 14, 15, 16, 17, 18, 19, or 20 ng/mL, or any concentration within a range defined by any two of the aforementioned concentrations; or
the BPE is at a concentration of about 5, 10, 20, 30, 40, 50, 60, 70, 80, 90, or 100 μg/mL, or any concentration within a range defined by any two of the aforementioned concentrations, or both.

19. The cell culture of any one of alternatives 15-18, further comprising a tissue culture container.

20. The cell culture of alternative 19, wherein the tissue culture container is coated with an extracellular matrix or a component thereof.

21. The cell culture of alternative 20, wherein the extracellular matrix or a component thereof is derived from human.

22. The cell culture of alternative 20 or 21, wherein the extracellular matrix or a component thereof comprises human collagen type IV.

23. The cell culture of any one of alternatives 20-22, wherein the extracellular matrix or a component thereof does not comprise rat collage type I matrix or Matrigel.

24. The cell culture of any one of alternatives 15-23, further comprising a ROCK inhibitor.

25. An in vitro cell culture comprising:
a population of anterior foregut cells treated with an EGF pathway activator, a BMP pathway inhibitor, an FGF pathway activator, or growth supplement, or any combination thereof.

26. The cell culture of alternative 25, further comprising a growth medium, such as RPMI, optionally with FBS, such as 0%, 0.1%, 0.2%, 0.3%, 0.4%, or 0.5% FBS, or any percentage of FBS within a range defined by any two of the aforementioned percentages.

27. The cell culture of alternative 25 or 26, further comprising a tissue culture container.

28. The esophageal raft culture of any one of alternatives 1-14, or the cell culture of any one of alternatives 15-27, wherein the esophageal raft culture or cell culture has been grown for at least 1, 2, 3, 4, 5, 6, 7, or 8 days.

29. The esophageal raft culture or the cell culture of alternative 28, wherein the esophageal raft culture or the cell culture is derived from human induced pluripotent stem cells.

30. The esophageal raft culture or the cell culture of alternative 28 or 29, wherein the esophageal raft culture or the cell culture is not derived from a spheroid or organoid.

31. A method of producing an esophageal raft culture, comprising:
(a) contacting anterior foregut cells with an EGF pathway activator, a BMP pathway inhibitor, an FGF pathway activator, or growth supplement, or any combination thereof to differentiate the anterior foregut cells into dorsal anterior foregut cells;
(b) dissociating the dorsal anterior foregut cells from step (a) into single cells;
(c) culturing the dorsal anterior foregut cells in a first tissue culture container to differentiate the dorsal anterior foregut cells to esophageal progenitor cells;
(d) dissociating the esophageal progenitor cells from step (c) into single cells;
(e) culturing the esophageal progenitor cells in an insert member,
wherein the insert member is positioned within a second tissue culture container,
wherein the insert member comprises a surface that is permeable to a growth medium but not cells; and
wherein the insert member and second tissue culture container each contain an amount of growth medium such that the esophageal progenitor cells are fully submerged in the growth medium; and
(f) culturing the esophageal progenitor cells in the insert member, wherein the insert member does not contain growth medium and the second tissue culture container contains an amount of growth medium such that the esophageal progenitor cells are partially submerged in the growth medium.

32. The method of alternative 31, wherein the esophageal progenitor cells are dissociated using a dissociation enzyme, such as trypsin, chymotrypsin, collagenase, elastase, or Accutase.

33. The method of alternative 31 or 32, wherein the first tissue culture container and/or the second tissue culture container are coated with an extracellular matrix or a component thereof.

34. The method of alternative 33, wherein the extracellular matrix or a component thereof is derived from human.

35. The method of alternative 33 or 34, wherein the extracellular matrix or a component thereof comprises human collagen type IV.

36. The method of any one of alternatives 33-35, wherein the extracellular matrix or a component thereof does not comprise rat collagen type I matrix or Matrigel.

37. The method of any one of alternatives 31-36, wherein the contacting step of (a) takes place over at least 1, 2, 3, 4, or 5 days.

38. The method of any one of alternatives 31-37, wherein the culturing step of (c) takes place over at least 1, 2, 3, 4, or 5 days.

39. The method of any one of alternatives 31-38, wherein the culturing step of (e) takes place over at least 2, 3, 4, 5, 6, 7, or 8 days.

40. The method of any one of alternatives 31-39, wherein the culturing step of (f) takes place over at least 10, 11, 12, 13, 14, 15, 16, 17, 18, 19, or 20 days.

41. The method of any one of alternatives 31-40, wherein the dorsal anterior foregut cells of step (c) are cultured with an EGF pathway activator, BPE, a ROCK inhibitor, or any combination thereof.

42. The method of any one of alternatives 31-41, wherein the esophageal progenitor cells of step (e) are cultured with an EGF pathway activator, a ROCK inhibitor, a SMAD inhibitor, or any combination thereof in the growth medium of the insert member and EGF in the growth medium of the second tissue culture container.

43. The method of any one of alternatives 31-42, wherein the esophageal progenitor cells of step (f) are cultured with an EGF pathway activator in the growth medium of the second tissue culture container.

44. The method of any one of alternatives 31-43, wherein the anterior foregut cells are derived from human induced pluripotent stem cells.

45. The method of any one of alternatives 31-44, wherein the anterior foregut cells are derived from definitive endoderm cells, wherein the definitive endoderm cells are derived from human induced pluripotent stem cells.

46. The method of alternative 45, wherein the definitive endoderm cells have been treated with Wnt3a, FGF4, Noggin, or RA, or any combination thereof.

47. The method of alternative 45 or 46, wherein the definitive endoderm cells are treated for 1, 2, 3, 4, or 5 days.

48. The method of any one of alternatives 44-47, wherein the human induced pluripotent stem cells are treated with BMP4 and/or Activin A.

49. The method of any one of alternatives 44-48, wherein the human induced pluripotent stem cells are treated for 1, 2, 3, 4, or 5 days.

50. The method of any one of alternatives 31-49, further comprising:
contacting human induced pluripotent stem cells with BMP4 and/or Activin A to differentiate the human induced pluripotent stem cells to definitive endoderm cells; and
contacting the definitive endoderm cells with Wnt, FGF4, Noggin, or RA, or any combination thereof to differentiate the definitive endoderm cells to the anterior foregut cells of step (a).

51. The method of alternative 50, wherein the human induced pluripotent stem cells are contacted for 1, 2, 3, 4 or 5 days.

52. The method of alternative 50 or 51, wherein the definitive endoderm cells are contacted for 1, 2, 3, 4, or 5 days.

53. An in vitro esophageal raft cell composition comprising:
a stratified squamous epithelium layer comprising a suprabasal layer and a basal layer; and
a mesenchyme layer comprising muscle fibers;
wherein the stratified squamous epithelium is E-cadherin⁺, the suprabasal layer is KRT13⁺ and KRT8⁺, and the basal layer is SOX2⁺, P63⁺, and KRT5⁺; and
wherein the mesenchyme layer is FOXF1⁺, NKX6-1⁺, and vimentin⁺, and the muscle fibers are desmin⁺.

54. An in vitro cell composition comprising:
a population of dorsal anterior foregut cells derived from anterior foregut cells treated with an EGF pathway activator, a BMP pathway inhibitor, an FGF pathway activator, or growth supplement, or any combination thereof.

55. An in vitro cell composition comprising:
a population of anterior foregut cells treated with an EGF pathway activator, a BMP pathway inhibitor, an FGF pathway activator, or growth supplement, or any combination thereof.

56. The esophageal raft cell composition of any one of the preceding alternatives, wherein the esophageal raft cell composition has a thickness of about 1, 5, 10, 20, 30, 40, 50, 60, 70, 80, 90, 100, 200, 300, 400, or 500 μm, or any thickness within a range defined by any two of the aforementioned thicknesses.

57. The esophageal raft cell composition of any one of the preceding alternatives, wherein the esophageal raft cell composition has a thickness of about 150, 200, 250, 300, 350, 400, 450, or 500 μm, or any thickness within a range defined by any two of the aforementioned thicknesses.

58. The esophageal raft cell composition of any one of the preceding alternatives, wherein the esophageal raft composition has a surface area of about 0.1, 0.5, 1, 5, 10, 15, 20, 25, 30, 40, 50, 60, 70, 80, 90, or 100 cm², or any surface area within a range defined by any two of the aforementioned surface areas.

59. The esophageal raft cell composition of any one of the preceding alternatives, wherein the esophageal raft composition has a surface area of about 0.1, 0.5, 1, 1.5, or 2 cm², or any surface area within a range defined by any two of the aforementioned surface areas.

60. The esophageal raft cell composition of any one of the preceding alternatives, wherein the esophageal raft composition has a volume of about 10⁻⁵, 10⁻⁴, 10⁻³, 10⁻², 10⁻¹, 1, 5 or 10 cm³, or any volume within a range defined by any two of the aforementioned volumes.

61. The esophageal raft cell composition of any one of the preceding alternatives, wherein the stratified squamous epithelium layer has a thickness of about 1, 5, 10, 20, 30, 40, 50, 60, 70, 80, 90, 100, 200, 300, 400, or 500 μm, or any thickness within a range defined by any two of the aforementioned thicknesses.

62. The esophageal raft cell composition of any one of the preceding alternatives, wherein the stratified squamous epithelium layer has a thickness of about 50, 60, 70, 80, 90, 100, 110, 120, 130, 140, 150, 160, 170, 180, 190, 200, 210, 220, 230, 240, or 250 μm, or any thickness within a range defined by any two of the aforementioned thicknesses.

63. The esophageal raft cell composition of any one of the preceding alternatives, wherein the suprabasal layer has a thickness of about 1, 5, 10, 20, 30, 40, 50, 60, 70, 80, 90, 100, 200, 300, 400, or 500 μm, or any thickness within a range defined by any two of the aforementioned thicknesses.

64. The esophageal raft cell composition of any one of the preceding alternatives, wherein the suprabasal layer has a thickness of about 80, 90, 100, 110, 120, 130, 140, 150, 160, 170, 180, 190, or 200 μm, or any thickness within a range defined by any two of the aforementioned thicknesses.

65. The esophageal raft cell composition of any one of the preceding alternatives, wherein the basal layer has a thickness of about 1, 5, 10, 20, 30, 40, 50, 60, 70, 80, 90, 100, 200, 300, 400, or 500 μm, or any thickness within a range defined by any two of the aforementioned thicknesses.

66. The esophageal raft cell composition of any one of the preceding alternatives, wherein the basal layer has a thickness of about 10, 20, 30, 40, 50, 60, 70, 80, 90, or 100 μm, or any thickness within a range defined by any two of the aforementioned thicknesses.

67. The esophageal raft cell composition of any one of the preceding alternatives, wherein the mesenchyme layer has a thickness of about 1, 5, 10, 20, 30, 40, 50, 60, 70, 80, 90, 100, 200, 300, 400, or 500 μm, or any thickness within a range defined by any two of the aforementioned thicknesses.

68. The esophageal raft cell composition of any one of the preceding alternatives, wherein the mesenchyme layer has a thickness of about 100, 150, 200, 250, 300, 350, or 400 μm, or any thickness within a range defined by any two of the aforementioned thicknesses.

69. The esophageal raft culture, cell culture, method, or esophageal raft cell composition of any one of the preceding alternatives, wherein the EGF pathway activator comprises EGF, TGF-alpha, AR, BTC, HB-EGF, EPR, tomoregulin, NRG-1, NRG-2, NRG-3, or NRG-4, or any combination thereof.

70. The esophageal raft culture, cell culture, method, or esophageal raft cell composition of any one of the preceding alternatives, wherein the EGF pathway activator is EGF.

71. The esophageal raft culture, cell culture, method, or esophageal raft cell composition of any one of the preceding alternatives, wherein the EGF pathway activator is provided at a concentration of about 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations.

72. The esophageal raft culture, cell culture, method, or esophageal raft cell composition of any one of the preceding alternatives, wherein the EGF pathway activator is provided at a concentration of 100 ng/mL or about 100 ng/mL.

73. The esophageal raft culture, cell culture, method, or esophageal raft cell composition of any one of the preceding alternatives, wherein the BMP pathway inhibitor comprises Noggin, RepSox, LY364947, LDN193189, SB431542, or any combination thereof.

74. The esophageal raft culture, cell culture, method, or esophageal raft cell composition of any one of the preceding alternatives, wherein the BMP pathway inhibitor is Noggin

75. The esophageal raft culture, cell culture, method, or esophageal raft cell composition of any one of the preceding alternatives, wherein the BMP pathway inhibitor is provided at a concentration of about 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations.

76. The esophageal raft culture, cell culture, method, or esophageal raft cell composition of any one of the preceding alternatives, wherein the BMP pathway inhibitor is provided at a concentration of 200 ng/mL or about 200 ng/mL.

77. The esophageal raft culture, cell culture, method, or esophageal raft cell composition of any one of the preceding alternatives, wherein the FGF pathway activator comprises FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, or FGF23, or any combination thereof.

78. The esophageal raft culture, cell culture, method, or esophageal raft cell composition of any one of the preceding alternatives, wherein the FGF pathway activator is FGF10.

79. The esophageal raft culture, cell culture, method, or esophageal raft cell composition of any one of the preceding alternatives, wherein the FGF pathway activator is provided at a concentration of about 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations.

80. The esophageal raft culture, cell culture, method, or esophageal raft cell composition of any one of the preceding alternatives, wherein the FGF pathway activator is provided at a concentration of 50 ng/mL or about 50 ng/mL.

81. The esophageal raft culture, cell culture, method, or esophageal raft cell composition of any one of the preceding alternatives, wherein the growth supplement is a serum-free growth supplement.

82. The esophageal raft culture, cell culture, method, or esophageal raft cell composition of any one of the preceding alternatives, wherein the growth supplement is CultureOne supplement.

83. The esophageal raft culture, cell culture, method, or esophageal raft cell composition of any one of the preceding alternatives, wherein the growth supplement is provided at a concentration of 1× or about 1×.

84. The esophageal raft culture, cell culture, method, or esophageal raft cell composition of any one of the preceding alternatives, wherein the ROCK inhibitor comprises Y-27632, Y-30141, Y-39983, Ki-23095, SLx-2119, thiazovivin, azaindole 1, fasudil, ripasudil, netarsudil, RKI-1447, or GSK429286A, or any combination thereof.

85. The esophageal raft culture, cell culture, method, or esophageal raft cell composition of any one of the preceding alternatives, wherein the ROCK inhibitor is Y-27632.

86. The esophageal raft culture, cell culture, method, or esophageal raft cell composition of any one of the preceding alternatives, wherein the ROCK inhibitor is provided at a concentration of about 1, 2, 3, 4, 5, 6, 7, 8, 9, 10, 11, 12, 13, 14, 15, 16, 17, 18, 19, or 20 μM, or any concentration within a range defined by any two of the aforementioned concentrations.

87. The esophageal raft culture, cell culture, method, or esophageal raft cell composition of any one of the preceding alternatives, wherein the ROCK inhibitor is provided at a concentration of 10 μM or about 10 μM.

88. The esophageal raft culture, cell culture, method, or esophageal raft cell composition of any one of the preceding alternatives, wherein the SMAD inhibitor comprises A-83-01, DMH1, RepSox, LY365947, LY2109761, LY364947, SB431542, SB525334, SB505125, galunisertib, GW788388, LDN-193189, LDN-212854, hesperetin, or any combination thereof.

89. The esophageal raft culture, cell culture, method, or esophageal raft cell composition of any one of the preceding alternatives, wherein the SMAD inhibitor is DMH1 and A-83-01.

90. The esophageal raft culture, cell culture, method, or esophageal raft cell composition of any one of the preceding alternatives, wherein the SMAD inhibitor is provided at a concentration of about 0.1, 0.5, 1, 2, 3, 4, 5, 6, 7, 8, 9, or 10 μM, or any concentration within a range defined by any two of the aforementioned concentrations.

91. The esophageal raft culture, cell culture, method, or esophageal raft cell composition of any one of the preceding alternatives, wherein the SMAD inhibitor is provided at a concentration of 1 μM or about 1 μM.

Additional embodiments of the present disclosure provided herein are described by way of the following alternatively numbered alternatives:

1. An in vitro esophageal raft culture comprising:
a stratified squamous epithelium layer comprising a suprabasal layer and a basal layer; and
a mesenchyme layer comprising muscle fibers;
wherein the stratified squamous epithelium is E-cadherin⁺, the suprabasal layer is KRT13⁺ and KRT8⁺, and the basal layer is SOX2⁺, P63⁺, and KRT5⁺; and
wherein the mesenchyme layer is FOXF1⁺, NKX6-1⁺, and vimentin⁺, and the muscle fibers are desmin⁺.

2. The esophageal raft culture of any one of the preceding alternatives, wherein the esophageal raft culture lacks a lamina propria layer, or has a reduced lamina propria layer compared to esophageal tissue from an adult animal of the same species as the raft culture.

3. The esophageal raft culture of any one of the preceding alternatives, further comprising a growth medium, optionally DMEM/F12.

4. The esophageal raft culture of any one of the preceding alternatives,
wherein the esophageal raft culture is located in, and/or on a surface of, an insert member comprising a surface that is permeable to the growth medium but not cells, and the insert member is positioned within a tissue culture container; and
optionally wherein the esophageal raft culture is positioned on the surface that is permeable to the growth medium but not cells.

5. The esophageal raft culture of alternative 4, wherein at least a portion of the insert member, optionally the surface that is permeable to the growth medium but not the cells, is coated with an extracellular matrix or a component thereof.

6. The esophageal raft culture of alternative 5, wherein the extracellular matrix or a component thereof is derived from human.

7. The esophageal raft culture of alternative 5 or 6, wherein the extracellular matrix or a component thereof comprises human collagen type IV.

8. The esophageal raft culture of any one of alternatives 5-7, wherein the extracellular matrix or a component thereof does not comprise rat collage type I matrix or Matrigel.

9. The esophageal raft culture of any one of alternatives 4-8, wherein the insert member and/or tissue culture container contain an amount of growth medium such that the esophageal raft culture is fully submerged in the growth medium.

10. The esophageal raft culture of alternative 9, wherein the growth medium contained within the insert member further comprises an EGF pathway activator, a ROCK inhibitor, a SMAD inhibitor, or any combination thereof, and the growth medium contained within the tissue culture container comprises an EGF pathway activator.

11. The esophageal raft culture of any one of alternatives 4-8, wherein the tissue culture container and/or insert member contain an amount of growth medium such that the esophageal raft culture is only partially submerged in the growth medium,
wherein the stratified squamous epithelium is only partially submerged or not submerged in the growth medium and forms and/or is located at an air-liquid interface.

12. The esophageal raft culture of alternative 11, wherein the growth medium contained within the tissue culture container comprises an EGF pathway activator.

13. The esophageal raft culture of any one of alternatives 4-12, wherein the surface that is permeable of the insert member comprises a pore size that is, is about, is at least, is at least about, is not more than, or is not more than about, 0.1, 0.5, 1, 2, 3, 4, 5, 6, 7, 8, 9, or 10 μm, or any pore size within a range defined by any two of the aforementioned sizes.

14. The esophageal raft culture of any one of alternatives 4-13, wherein the surface that is permeable of the insert member comprises a pore size of 3 μm.

15. The esophageal raft culture of any one of alternatives 1-14, wherein the esophageal raft culture is effectively free of neuronal progenitor cells and/or βIII-tubulin+ neuronal cells.

16. The esophageal raft culture of any one of alternatives 1-14, wherein the esophageal raft culture further comprises enteric neural crest cells (ENCCs), neuronal progenitor cells and/or βIII-tubulin+ neuronal cells, such that the esophageal raft culture is an innervated esophageal raft culture, optionally wherein the neuronal progenitor cells are SOX10+.

17. The esophageal raft culture of any one of alternatives 1-16, wherein the esophageal raft culture does not have vascularization, blood vessels, and/or endothelial cells.

18. An in vitro cell culture comprising:
a population of esophageal progenitor cells derived from dorsal anterior foregut cells which have been treated with an EGF pathway activator, a BMP pathway inhibitor, an FGF pathway activator, or any combination thereof.

19. The cell culture of alternative 18, wherein the dorsal anterior foregut cells have also been treated with a neuronal progenitor inhibitor, optionally CultureOne supplement or cytarabine.

20. The cell culture of alternative 18 or 19, further comprising a growth medium, optionally a serum free medium, optionally Keratinocyte SFM.

21. The cell culture of alternative 20, wherein the growth medium comprises an EGF pathway activator or bovine pituitary extract (BPE), or both.

22. The cell culture of alternative 21, wherein:
the EGF pathway activator is at a concentration of about 1, 2, 3, 4, 5, 6, 7, 8, 9, 10, 11, 12, 13, 14, 15, 16, 17, 18, 19, or 20 ng/mL, or any concentration within a range defined by any two of the aforementioned concentrations; or
the BPE is at a concentration of about 5, 10, 20, 30, 40, 50, 60, 70, 80, 90, or 100 μg/mL, or any concentration within a range defined by any two of the aforementioned concentrations, or both.

23. The cell culture of any one of alternatives 18-22, wherein the cell culture is located in, and/or on a surface of, a tissue culture container.

24. The cell culture of alternative 23, wherein at least a portion of the tissue culture container is coated with an extracellular matrix or a component thereof, and said population of esophageal progenitor cells are on or in contact with said portion.

25. The cell culture of alternative 24, wherein the extracellular matrix or a component thereof is derived from human.

26. The cell culture of alternative 24 or 25, wherein the extracellular matrix or a component thereof comprises human collagen type IV.

27. The cell culture of any one of alternatives 24-26, wherein the extracellular matrix or a component thereof does not comprise rat collage type I matrix or Matrigel.

28. The cell culture of any one of alternatives 18-27, further comprising a ROCK inhibitor.

29. The cell culture of any one of alternatives 18-28, further comprising enteric neural crest cells.

30. An in vitro cell culture comprising:
a population of anterior foregut cells treated with an EGF pathway activator, a BMP pathway inhibitor, an FGF pathway activator, or any combination thereof.

31. The cell culture of alternative 30, wherein the anterior foregut cells are further treated with a neuronal progenitor inhibitor, optionally CultureOne supplement or cytarabine.

32. The cell culture of alternative 30 or 31, further comprising a growth medium, optionally RPMI, optionally with FBS, optionally 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% FBS, or any percentage of FBS within a range defined by any two of the aforementioned percentages.

33. The cell culture of any one of alternatives 30-32, wherein said cell culture is located in, and/or on a surface of a tissue culture container.

34. The esophageal raft culture of any one of alternatives 1-17, or the cell culture of any one of alternatives 18-33, wherein the esophageal raft culture or cell culture has been grown for at least 1, 2, 3, 4, 5, 6, 7, or 8 days.

35. The esophageal raft culture or the cell culture of alternative 34, wherein the esophageal raft culture or the cell culture have been derived from human induced pluripotent stem cells.

36. The esophageal raft culture or the cell culture of alternative 34 or 35, wherein the esophageal raft culture or the cell culture is not derived from a spheroid or organoid.

37. A method of producing an esophageal raft culture, comprising:
(a) contacting anterior foregut cells with an EGF pathway activator, a BMP pathway inhibitor, an FGF pathway activator, or any combination thereof to differentiate the anterior foregut cells into dorsal anterior foregut cells;
(b) dissociating the dorsal anterior foregut cells from step (a) into single cells;
(c) culturing the dorsal anterior foregut cells in a first tissue culture container to differentiate the dorsal anterior foregut cells to esophageal progenitor cells;
(d) dissociating the esophageal progenitor cells from step (c) into single cells;
(e) culturing the esophageal progenitor cells in, and/or on a surface of, an insert member,
wherein the insert member is positioned within a second tissue culture container,
wherein the insert member comprises a surface that is permeable to a growth medium but not cells; and
wherein the insert member and second tissue culture container each contain an amount of growth medium such that the esophageal progenitor cells are fully submerged in the growth medium; and
(f) culturing the esophageal progenitor cells in the insert member, wherein the second tissue culture container and/or insert member contains an amount of growth medium such that the esophageal progenitor cells are only partially submerged in the growth medium.

38. The method of alternative 37, wherein the anterior foregut cells are further contacted with a neuronal progenitor inhibitor, optionally CultureOne supplement or cytarabine.

39. The method of alternative 37 or 38, wherein the esophageal progenitor cells are dissociated using a dissociation enzyme, optionally trypsin, chymotrypsin, collagenase, elastase, or Accutase.

40. The method of any one of alternatives 37-39, wherein at least a portion of the first tissue culture container and/or the second tissue culture container are coated with an extracellular matrix or a component thereof.

41. The method of alternative 40, wherein the extracellular matrix or a component thereof is derived from human.

42. The method of alternative 40 or 41, wherein the extracellular matrix or a component thereof comprises human collagen type IV.

43. The method of any one of alternatives 40-42, wherein the extracellular matrix or a component thereof does not comprise rat collagen type I matrix or Matrigel.

44. The method of any one of alternatives 37-43, wherein the contacting step of (a) takes place over at least 1, 2, 3, 4, or 5 days.

45. The method of any one of alternatives 37-44, wherein the culturing step of (c) takes place over at least 1, 2, 3, 4, or 5 days.

46. The method of any one of alternatives 37-45, wherein the culturing step of (e) takes place over at least 2, 3, 4, 5, 6, 7, or 8 days.

47. The method of any one of alternatives 37-46, wherein the culturing step of (f) takes place over at least 10, 11, 12, 13, 14, 15, 16, 17, 18, 19, or 20 days.

48. The method of any one of alternatives 37-47, wherein the dorsal anterior foregut cells of step (c) are cultured with an EGF pathway activator, BPE, a ROCK inhibitor, or any combination thereof.

49. The method of any one of alternatives 37-48, wherein the esophageal progenitor cells of step (e) are cultured with an EGF pathway activator, a ROCK inhibitor, a SMAD inhibitor, or any combination thereof in the growth medium of the insert member and EGF in the growth medium of the second tissue culture container.

50. The method of any one of alternatives 37-49, wherein the esophageal progenitor cells of step (f) are cultured with an EGF pathway activator in the growth medium of the second tissue culture container.

51. The method of any one of alternatives 37-50, wherein the anterior foregut cells have been derived from human induced pluripotent stem cells.

52. The method of any one of alternatives 37-51, wherein the anterior foregut cells have been derived from definitive endoderm cells, wherein the definitive endoderm cells have been derived from human induced pluripotent stem cells.

53. The method of alternative 52, wherein the definitive endoderm cells have been treated with Wnt3a, FGF4, Noggin, or RA, or any combination thereof, to differentiate the definitive endoderm cells to anterior foregut cells.

54. The method of alternative 53, wherein the definitive endoderm cells have been further treated with a neuronal progenitor inhibitor, optionally CultureOne supplement or cytarabine.

55. The method of any one of alternatives 52-54, wherein the definitive endoderm cells have been treated for 1, 2, 3, 4, or 5 days.

56. The method of any one of alternatives 51-55, wherein the human induced pluripotent stem cells have been treated with BMP4 and/or Activin A to differentiate the human induced pluripotent stem cells to definitive endoderm cells.

57. The method of alternative 56, wherein the human induced pluripotent stem cells have been further treated with a neuronal progenitor inhibitor, optionally CultureOne supplement or cytarabine.

58. The method of any one of alternatives 51-57, wherein the human induced pluripotent stem cells have been treated for 1, 2, 3, 4, or 5 days.

59. The method of any one of alternatives 37-58, further comprising:
contacting human induced pluripotent stem cells with BMP4 and/or Activin A to differentiate the human induced pluripotent stem cells to definitive endoderm cells; and
contacting the definitive endoderm cells with Wnt, FGF4, Noggin, or RA, or any combination thereof to differentiate the definitive endoderm cells to the anterior foregut cells of step (a).

60. The method of alternative 59, wherein the human induced pluripotent stem cells and/or the definitive endoderm cells are further contacted with a neuronal progenitor inhibitor, optionally CultureOne supplement or cytarabine.

61. The method of alternative 59 or 60, wherein the human induced pluripotent stem cells are contacted for 1, 2, 3, 4 or 5 days.

62. The method of any one of alternatives 59-61, wherein the definitive endoderm cells are contacted for 1, 2, 3, 4, or 5 days.

63. The method of any one of alternatives 37-62, wherein the esophageal raft culture is effectively free of neuronal progenitor cells and/or βIII-tubulin+ neuronal cells.

64. The method of any one of alternatives 37-63, further comprising combining the dissociated esophageal progenitor cells of step (d) with enteric neural crest cells (ENCCs) and culturing the combined esophageal progenitor cells and ENCCs according to steps (e) and (f) to produce an innervated esophageal raft culture.

65. The method of alternative 64, wherein the innervated esophageal raft culture comprises enteric neural crest cells (ENCCs), neuronal progenitor cells and/or βIII-tubulin+neuronal cells, optionally wherein the neuronal progenitor cells are SOX10+.

66. The method of any one of alternatives 37-65, wherein the esophageal raft culture does not comprise vascularization, blood vessels, and/or endothelial cells.

67. An in vitro cell composition comprising:
a population of dorsal anterior foregut cells derived from anterior foregut cells treated with an EGF pathway activator, a BMP pathway inhibitor, an FGF pathway activator, or any combination thereof.

68. An in vitro cell composition comprising:
a population of anterior foregut cells treated with an EGF pathway activator, a BMP pathway inhibitor, an FGF pathway activator, or any combination thereof.

69. The cell composition of alternative 68, wherein the population of anterior foregut cells is further treated with a neuronal progenitor inhibitor, optionally CultureOne supplement or cytarabine.

70. An in vitro esophageal raft cell composition comprising:
a stratified squamous epithelium layer comprising a suprabasal layer and a basal layer; and
a mesenchyme layer comprising muscle fibers;
wherein the stratified squamous epithelium is E-cadherin⁺, the suprabasal layer is KRT13⁺ and KRT8⁺, and the basal layer is SOX2⁺, P63⁺, and KRT5⁺; and
wherein the mesenchyme layer is FOXF1⁺, NKX6-1⁺, and vimentin⁺, and the muscle fibers are desmin⁺.

71. The esophageal raft cell composition of alternative 70, wherein the esophageal raft cell composition is effectively free of neuronal progenitor cells and/or βIII-tubulin+ neuronal cells.

72. The esophageal raft cell composition of alternative 70, wherein the esophageal raft cell composition further comprises enteric neural crest cells (ENCCs), neuronal progenitor cells and/or βIII-tubulin+ neuronal cells, such that the esophageal raft culture is an innervated esophageal raft culture, optionally wherein the neuronal progenitor cells are SOX10+.

73. The esophageal raft cell composition of any one of alternatives 70-72, wherein the esophageal raft cell composition does not comprise vascularization, blood vessels, and/or endothelial cells.

74. The esophageal raft cell composition of any one of the preceding alternatives, wherein the esophageal raft cell composition has a thickness of about 1, 5, 10, 20, 30, 40, 50, 60, 70, 80, 90, 100, 200, 300, 400, or 500 μm, or any thickness within a range defined by any two of the aforementioned thicknesses.

75. The esophageal raft cell composition of any one of the preceding alternatives, wherein the esophageal raft cell composition has a thickness of about 150, 200, 250, 300, 350, 400, 450, or 500 μm, or any thickness within a range defined by any two of the aforementioned thicknesses.

76. The esophageal raft cell composition of any one of the preceding alternatives, wherein the esophageal raft composition has a surface area of about 0.1, 0.5, 1, 5, 10, 15, 20, 25, 30, 40, 50, 60, 70, 80, 90, or 100 cm², or any surface area within a range defined by any two of the aforementioned surface areas.

77. The esophageal raft cell composition of any one of the preceding alternatives, wherein the esophageal raft composition has a surface area of about 0.1, 0.5, 1, 1.5, or 2 cm², or any surface area within a range defined by any two of the aforementioned surface areas.

78. The esophageal raft cell composition of any one of the preceding alternatives, wherein the esophageal raft composition has a volume of about 10⁻⁵, 10⁻⁴, 10⁻³, 10⁻², 10⁻¹, 1, 5 or 10 cm³, or any volume within a range defined by any two of the aforementioned volumes.

79. The esophageal raft cell composition of any one of the preceding alternatives, wherein the stratified squamous epithelium layer has a thickness of about 1, 5, 10, 20, 30, 40, 50, 60, 70, 80, 90, 100, 200, 300, 400, or 500 μm, or any thickness within a range defined by any two of the aforementioned thicknesses.

80. The esophageal raft cell composition of any one of the preceding alternatives, wherein the stratified squamous epithelium layer has a thickness of about 50, 60, 70, 80, 90, 100, 110, 120, 130, 140, 150, 160, 170, 180, 190, 200, 210, 220, 230, 240, or 250 μm, or any thickness within a range defined by any two of the aforementioned thicknesses.

81. The esophageal raft cell composition of any one of the preceding alternatives, wherein the suprabasal layer has a thickness of about 1, 5, 10, 20, 30, 40, 50, 60, 70, 80, 90, 100, 200, 300, 400, or 500 μm, or any thickness within a range defined by any two of the aforementioned thicknesses.

82. The esophageal raft cell composition of any one of the preceding alternatives, wherein the suprabasal layer has a thickness of about 80, 90, 100, 110, 120, 130, 140, 150, 160, 170, 180, 190, or 200 μm, or any thickness within a range defined by any two of the aforementioned thicknesses.

83. The esophageal raft cell composition of any one of the preceding alternatives, wherein the basal layer has a thickness of about 1, 5, 10, 20, 30, 40, 50, 60, 70, 80, 90, 100, 200, 300, 400, or 500 μm, or any thickness within a range defined by any two of the aforementioned thicknesses.

84. The esophageal raft cell composition of any one of the preceding alternatives, wherein the basal layer has a thickness of about 10, 20, 30, 40, 50, 60, 70, 80, 90, or 100 μm, or any thickness within a range defined by any two of the aforementioned thicknesses.

85. The esophageal raft cell composition of any one of the preceding alternatives, wherein the mesenchyme layer has a thickness of about 1, 5, 10, 20, 30, 40, 50, 60, 70, 80, 90, 100, 200, 300, 400, or 500 μm, or any thickness within a range defined by any two of the aforementioned thicknesses.

86. The esophageal raft cell composition of any one of the preceding alternatives, wherein the mesenchyme layer has a thickness of about 100, 150, 200, 250, 300, 350, or 400 μm, or any thickness within a range defined by any two of the aforementioned thicknesses.

87. The esophageal raft culture, cell culture, method, or esophageal raft cell composition of any one of the preceding alternatives, wherein the EGF pathway activator comprises EGF, TGF-alpha, AR, BTC, HB-EGF, EPR, tomoregulin, NRG-1, NRG-2, NRG-3, or NRG-4, or any combination thereof.

88. The esophageal raft culture, cell culture, method, or esophageal raft cell composition of any one of the preceding alternatives, wherein the EGF pathway activator is EGF.

89. The esophageal raft culture, cell culture, method, or esophageal raft cell composition of any one of the preceding alternatives, wherein the EGF pathway activator is provided at a concentration of about 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations.

90. The esophageal raft culture, cell culture, method, or esophageal raft cell composition of any one of the preceding alternatives, wherein the EGF pathway activator is provided at a concentration of 100 ng/mL or about 100 ng/mL.

91. The esophageal raft culture, cell culture, method, or esophageal raft cell composition of any one of the preceding alternatives, wherein the BMP pathway inhibitor comprises Noggin, RepSox, LY364947, LDN193189, SB431542, or any combination thereof.

92. The esophageal raft culture, cell culture, method, or esophageal raft cell composition of any one of the preceding alternatives, wherein the BMP pathway inhibitor is Noggin.

93. The esophageal raft culture, cell culture, method, or esophageal raft cell composition of any one of the preceding alternatives, wherein the BMP pathway inhibitor is provided at a concentration of about 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations.

94. The esophageal raft culture, cell culture, method, or esophageal raft cell composition of any one of the preceding alternatives, wherein the BMP pathway inhibitor is provided at a concentration of 200 ng/mL or about 200 ng/mL.

95. The esophageal raft culture, cell culture, method, or esophageal raft cell composition of any one of the preceding alternatives, wherein the FGF pathway activator comprises FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, or FGF23, or any combination thereof.

96. The esophageal raft culture, cell culture, method, or esophageal raft cell composition of any one of the preceding alternatives, wherein the FGF pathway activator is FGF10.

97. The esophageal raft culture, cell culture, method, or esophageal raft cell composition of any one of the preceding alternatives, wherein the FGF pathway activator is provided at a concentration of about 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations.

98. The esophageal raft culture, cell culture, method, or esophageal raft cell composition of any one of the preceding alternatives, wherein the FGF pathway activator is provided at a concentration of 50 ng/mL or about 50 ng/mL.

99. The esophageal raft culture, cell culture, method, or esophageal raft cell composition of any one of the preceding alternatives, wherein the ROCK inhibitor comprises Y-27632, Y-30141, Y-39983, Ki-23095, SLx-2119, thiazovivin, azaindole 1, fasudil, ripasudil, netarsudil, RKI-1447, or GSK429286A, or any combination thereof.

100. The esophageal raft culture, cell culture, method, or esophageal raft cell composition of any one of the preceding alternatives, wherein the ROCK inhibitor is Y-27632.

101. The esophageal raft culture, cell culture, method, or esophageal raft cell composition of any one of the preceding alternatives, wherein the ROCK inhibitor is provided at a concentration of about 1, 2, 3, 4, 5, 6, 7, 8, 9, 10, 11, 12, 13, 14, 15, 16, 17, 18, 19, or 20 μM, or any concentration within a range defined by any two of the aforementioned concentrations.

102. The esophageal raft culture, cell culture, method, or esophageal raft cell composition of any one of the preceding alternatives, wherein the ROCK inhibitor is provided at a concentration of 10 μM or about 10 μM.

103. The esophageal raft culture, cell culture, method, or esophageal raft cell composition of any one of the preceding alternatives, wherein the SMAD inhibitor comprises A-83-01, DMH1, RepSox, LY365947, LY2109761, LY364947, SB431542, SB525334, SB505125, galunisertib, GW788388, LDN-193189, LDN-212854, hesperetin, or any combination thereof.

104. The esophageal raft culture, cell culture, method, or esophageal raft cell composition of any one of the preceding alternatives, wherein the SMAD inhibitor is DMH1 and A-83-01.

105. The esophageal raft culture, cell culture, method, or esophageal raft cell composition of any one of the preceding alternatives, wherein the SMAD inhibitor is provided at a concentration of about 0.1, 0.5, 1, 2, 3, 4, 5, 6, 7, 8, 9, or 10 μM, or any concentration within a range defined by any two of the aforementioned concentrations.

106. The esophageal raft culture, cell culture, method, or esophageal raft cell composition of any one of the preceding alternatives, wherein the SMAD inhibitor is provided at a concentration of 1 μM or about 1 μM.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1 depicts an embodiment of a schematic to produce esophageal raft cultures described herein.

FIG. 2 depicts an embodiment of esophageal raft cultures comprising epithelium and mesenchyme. Apical layer of the raft culture expresses esophagus epithelium markers SOX2 and P63 as well as the epithelium general marker E-cadherin (Ecad) (panel A). The epithelium is stratified squamous epithelium expressing basal (KRT5) and suprabasal (KRT13 and KRT8) layer markers in distinct layers (panel B). The two distinct layers can be identified in hematoxylin/eosin staining (panel C). Raft culture basal layer expresses mesenchyme markers FOXF1, NKX6-1 (panel D) and Vimentin (panel E). The mesenchyme expresses markers of differentiated muscle fibers (Desmin) (panel F). Cell nuclei are labeled with DAPI as a general marker. Scale bar is 100 μm. Also shown is a schematic of the esophageal raft cultures, with distinct layers of epithelium and mesenchyme with characteristic markers (panel G).

FIG. 3A depicts an embodiment of a schematic to produce esophageal raft cultures described herein, where an optional neuronal progenitor inhibitor (e.g. CultureOne supplement [Cult1]) is added from days 0-9. The addition of a neuronal progenitor inhibitor prevents expansion of neuronal cell types during differentiation of the esophageal raft culture, which might not be desirable for some purposes.

FIG. 3B depicts an embodiment of immunofluorescence images showing esophageal raft cultures differentiated with the use of a neuronal progenitor inhibitor (e.g. CultureOne supplement) from either days 0-9 or days 6-9. The esophageal raft cultures were stained for 1) hematoxylin & eosin, 2) SOX2, 3) E-cadherin (Ecad), 4) merge of SOX, Ecad, and DAPI, or 5) βIII-tubulin and DAPI. Dashed line indicates the mesenchyme-epithelium boundary. Arrows indicate the presence of neuronal cell types in the mesenchyme of the raft cultures treated with CultureOne only from day 6 onwards. These neuronal cell types are not seen in the cultures grown with CultureOne from days 0-9. Neuronal cell types are indicated by expression of SOX2 and βIII-tubulin (arrows) in the mesenchymal layer.

FIG. 4A depicts an embodiment of a schematic to produce innervated esophageal raft cultures. Esophageal progenitor cells and enteric neural crest cells (ENCCs) are separately differentiated from hPSC and co-cultured on cell inserts to generate esophageal raft cultures with mesenchyme innervated by enteric neurons.

FIG. 4B depicts an embodiment of immunofluorescence images showing innervated esophageal raft cultures. The innervated esophageal raft cultures were stained from 1) SOX2, P63, Ecad, and DAPI, 2) KRT5, KRT13, and DAPI, 3) GFP (expressed by ENCCs), vimentin, KRT8, and DAPI, or 4) GFP (expressed by ENCCs), KRT8, and DAPI. ENCC-innervated esophageal raft cultures express esophageal epithelial markers SOX2, P63, KRT5, KRT13, and KRT8, and the general epithelium marker E-cadherin (Ecad). GFP-expressing ENCCs (arrows) innervates the esophageal raft culture mesenchyme, which is marked by vimentin expression. GFP-expressing ENCCs co-localize with the neural marker βIII-tubulin, indicating their differentiation into enteric neurons.

### DETAILED DESCRIPTION

Disclosed herein are iPSC-derived esophageal raft cultures and esophageal raft cell compositions. In some embodiments, the esophageal raft cultures and esophageal raft cell compositions described herein are produced from human iPSCs. In some embodiments, the esophageal raft cultures and esophageal raft cell compositions comprise esophageal epithelium and esophageal mesenchyme. In some embodiments, the esophageal raft cultures and esophageal raft cell compositions can be grown to exclude neuronal cell types by culturing with inhibitors of neuronal progenitors (i.e., compounds that inhibit growth and/or differentiation of neuronal progenitor cells and neuronal cell types). In some embodiments, the esophageal raft cultures and esophageal raft cell compositions can be grown to be innervated by culturing the esophageal raft cultures, or progenitors thereof, such as esophageal progenitor cells, with enteric neural crest cells (ENCCs), which differentiate into neuronal cell types. Also disclosed herein are methods of making the esophageal raft cultures and esophageal raft cell compositions. In some embodiments, the esophageal raft cultures and esophageal raft cell compositions are distinct from esophageal organoids. By manipulating factors that control embryonic organogenesis, in vitro methods have been developed to guide the stepwise differentiation of PSCs into embryonic germ layer lineages, and subsequently into specific cell types such as epithelial cells, mesenchymal cells, myocytes, neural cells, and vascular cells. Methods of producing esophageal organoids is explored in PCT Publication WO 2019/074793, hereby expressly incorporated by reference in its entirety. Additional methods of producing other types of organoids and intermediate cell types (e.g. definitive endoderm and anterior foregut spheroids) may be found in U.S. Pat. Nos. 9,719,068 and 10,174,289, and PCT Publications WO 2011/140411, WO 2015/183920, WO 2016/061464, WO 2017/192997, WO 2018/106628, WO 2018/200481, WO 2018/085615, WO 2018/085622, WO 2018/085623, WO 2018/226267, WO 2020/023245, each of which is hereby expressly incorporated by reference in its entirety. Methods of producing other types of organoids that are innervated using enteric neural crest cells (ENCCs) are explored in PCT Publication WO 2016/061464, which is hereby expressly incorporated by reference in its entirety.

The esophagus actively facilitates the passing of food from the oral cavity and pharynx to the stomach. It consists of a stratified squamous epithelium, mesenchyme, muscle layers, and an enteric nervous system to sense stretch and control peristalsis. Congenital diseases such as esophageal atresia are caused by gene mutations that result in luminal narrowing or discontinuity. Other diseases affect the esophagus later in life, such as esophageal carcinoma, eosinophilic esophagitis, achalasia and other motility disorders. Tracheal and esophageal disorders are prevalent in humans and are difficult to accurately model in mice. The need for improved esophageal and other gastrointestinal models is manifest.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “raft culture” as used herein has its plain and ordinary meaning as understood in light of the specification and refers to a three-dimensional cell culture comprising more than one cell type having cell organization and function that closely resembles native organ tissue. In some embodiments, as the name suggests, the culture is grown and maintained in an air-liquid interface where a portion of the culture is exposed to a gaseous or atmospheric environment while another portion is situated on or within a layer of liquid growth medium. Diffusion allows nutrients from the growth medium to access the exposed cells. The air-liquid interface encourages the differentiation of the raft culture into stratified epithelial layers, which are found in all organs, including those that are exposed to the environmental atmosphere in vivo, including the lungs, esophagus, and skin. In some embodiments described herein, the raft culture also comprises a mesenchymal layer or mesenchyme.

The term “insert member” as used herein has its plain and ordinary meaning as understood in light of the specification and refers to any construction or container having at least a surface on which cells can grow, where the surface or at least a portion thereof that is permeable to an aqueous medium but not cells, and which can be situated within a separate container or other construction such that the cells are exposed to the environment of both the insert member and the separate container or other construction (although the cells may be exposed to the environment of the separate container or other construction across the surface or at least the portion thereof that is permeable to an aqueous medium but not cells, and not necessarily in direct contact). A common example of an insert member as used herein are transwells, which are tissue culture containers that have a permeable surface and can be situated within a separate, generally larger volume tissue culture container, such that aqueous media can be contained within one or more of the internal volume of the transwell or the internal volume of the separate tissue culture container, such that exchange between the two aqueous media can occur across the permeable surface, and cells can be contacted on the permeable surface or at least a portion thereof such that the cells are exposed to the aqueous medium in the transwell and the aqueous medium in the separate container. However, alternative constructions of insert members are envisioned, such as those where the insert member is affixed to the separate container, and channels or other openings are available to access the internal volume of the separate container, or where either or both of the transwell or the separate container do not have a traditional internal volume, and the contact between the aqueous medium in the insert member and the separate container is done through alternative means such as microfluidic channels. As disclosed herein, cells can be grown on a surface or a portion thereof of an insert member within a separate container such as that the cells are only partially submerged (i.e. with little or no aqueous medium in the insert member) within an air-liquid interface.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof. Other methods of producing induced pluripotent stem cells as conventionally known in the art are also envisioned.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC), and induced pluripotent stem cells.

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UCO6 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “differentiation” or “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of target stem cells.

### Differentiation of PSCs and Definitive Endoderm

In some embodiments, PSCs, such as ESCs and iPSCs, undergo directed differentiation in a stepwise manner first into definitive endoderm (DE) then into anterior/foregut lineages, and then into esophageal tissue. In some embodiments, PSCs, such as ESCs and iPSCs, undergo directed differentiation in a non-stepwise manner where molecules (e.g., growth factors, ligands) for promoting DE formation and those for subsequent tissue formation are added at the same time.

The definitive endoderm gives rise to the gut tube. The anterior DE forms the foregut and its associated organs including the esophagus, lungs, stomach, liver and pancreas and the posterior DE forms the midgut and hindgut, which forms the small and large intestines and parts of the genitourinary system. Studies using mouse, chick and frog embryos suggest that establishing the anterior-posterior pattern in DE at the gastrula stage is a prerequisite for subsequent foregut and hindgut development. The Wnt and FGF signaling pathways are important for promoting either posterior endoderm/hindgut or anterior endoderm/foregut fate.

Methods of directing differentiation of DE into esophageal tissue in vitro has been explored in PCT Publication WO 2019/074793, hereby expressly incorporated by reference in its entirety. In some embodiments, directed differentiation is achieved by selectively activating certain signaling pathways in the iPSCs and/or DE cells. In some embodiments, the signaling pathways are those active in esophageal or gastrointestinal development, including but not limited to the EGF signaling pathway; Wnt signaling pathway; Wnt/APC signaling pathway; FGF signaling pathway; TGF-beta signaling pathway; BMP signaling pathway; Notch signaling pathway; Hedgehog signaling pathway; LKB signaling pathway; and Par polarity signaling pathway.

Methods for producing definitive endoderm from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce definitive endoderm. In some embodiments, human embryonic germ cells are used to produce definitive endoderm. In some embodiments, iPSCs are used to produce definitive endoderm. In some embodiments, human iPSCs (hiPSCs) are used to produce definitive endoderm.

In some embodiments, the embryonic stem cells or germ cells or iPSCs are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the embryonic stem cells or germ cells or iPSCs are treated with one or more small molecule compounds, activators, inhibitors, or growth factors at a concentration that is, is about, is at least, is at least about, is not more than, or is not more than about, 10 ng/mL, 20 ng/mL, 50 ng/mL, 75 ng/mL, 100 ng/mL, 120 ng/mL, 150 ng/mL, 200 ng/mL, 500 ng/mL, 1000 ng/mL, 1200 ng/mL, 1500 ng/mL, 2000 ng/mL, 5000 ng/mL, 7000 ng/mL, 10000 ng/mL, or 15000 ng/mL, or any concentration that is within a range defined by any two of the aforementioned concentrations, for example, 10 ng/mL to 15000 ng/mL, 100 ng/mL to 5000 ng/mL, 500 ng/mL to 2000 ng/mL, 10 ng/mL to 2000 ng/mL, or 1000 ng/mL to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs, germ cells, or iPSCs, or any downstream cell types, are cultured in growth media that supports the growth of stem cells. In some embodiments, the ESCs, germ cells, or iPSCs, or any downstream cell types, are cultured in stem cell growth media. In some embodiments, the stem cell growth media is a serum free medium, RPMI 1640, DMEM, DMEM/F12, Advanced DMEM/F12, or Keratinocyte SFM media. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

In some embodiments, definitive endoderm cells and hESCs are treated with one or more growth factors. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, Wnt3a or combinations of any of these growth factors.

In some embodiments, activin-induced definitive endoderm (DE) can further undergo anterior endoderm pattering, foregut specification and morphogenesis, dependent on FGF, Wnt, BMP, or retinoic acid, or any combination thereof, and an esophageal culture system that promoted esophageal growth, morphogenesis and cytodifferentiation. In some embodiments, human PSCs are efficiently directed to differentiate in vitro into esophageal epithelium and mesenchyme. It will be understood that molecules such as growth factors can be added to any stage of the development to promote a particular type of gastrointestinal tissue formation.

In some embodiments, directed differentiation is achieved by selectively activating and/or inhibiting certain signaling pathways in the PSCs, DE, or any downstream cell types. In some embodiments, the signaling pathways include but are not limited to the Wnt pathway; FGF pathway, BMP pathway; retinoic acid pathway; EGF pathway; Rho kinase (ROCK) pathway; or SMAD pathway, or any combination thereof. It will be understood by one of skill in the art that altering the concentration, expression or function of any one of the signaling pathways disclosed herein can drive differentiation in accordance with the present disclosure. In some embodiments, cellular constituents associated with the signaling pathways, for example, natural inhibitors, antagonists, activators, or agonists of the pathways can be used to result in inhibition or activation of the signaling pathways. In some embodiments, siRNA and/or shRNA targeting cellular constituents associated with the signaling pathways are used to inhibit or activate these pathways.

In some embodiments, pluripotent stem cells, definitive endoderm cells, anterior foregut cells, dorsal anterior foregut cells, esophageal progenitor cells, and/or esophageal raft cells are contacted with a Wnt pathway activator. In some embodiments, the Wnt pathway activator comprises a Wnt protein. In some embodiments, the Wnt protein comprises a recombinant Wnt protein. In some embodiments, the Wnt pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt pathway activator comprises a GSK3 signaling pathway inhibitor. In some embodiments, the Wnt pathway activator comprises CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the cells are not treated with a Wnt pathway activator. The Wnt pathway activators provided herein may be used in combination with any of the other growth factors, pathway activators, or pathway inhibitors described herein.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic, and FGF2 is also known as basic fibroblast growth factor (bFGF). Members FGF11, FGF12, FGF13, and FGF14, also known as FGF homologous factors 1-4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects.

In some embodiments, pluripotent stem cells, definitive endoderm cells, anterior foregut cells, dorsal anterior foregut cells, esophageal progenitor cells, and/or esophageal raft cells are contacted with an FGF pathway activator. In some embodiments, the FGF pathway activator comprises an FGF protein. In some embodiments, the FGF protein comprises a recombinant FGF protein. In some embodiments, the FGF pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF pathway activator. The FGF pathway activator provided herein may be used in combination with any of the other growth factors, pathway activators, or pathway inhibitors provided herein.

In some embodiments, pluripotent stem cells, definitive endoderm cells, anterior foregut cells, dorsal anterior foregut cells, esophageal progenitor cells, and/or esophageal raft cells are contacted with a bone morphogenetic protein (BMP) pathway activator or BMP pathway inhibitor. In some embodiments, the BMP pathway activator comprises a BMP protein. In some embodiments, the BMP protein is a recombinant BMP protein. In some embodiments, the BMP pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP pathway inhibitor comprises Noggin, RepSox, LY364947, LDN193189, SB431542, or any combination thereof. In some embodiments, the cells are not treated with a BMP pathway activator or BMP pathway inhibitor. The BMP pathway activator or BMP pathway inhibitor provided herein may be used in combination with any of the other growth factors, pathway activators, or pathway inhibitors provided herein.

In some embodiments, pluripotent stem cells, definitive endoderm cells, anterior foregut cells, dorsal anterior foregut cells, esophageal progenitor cells, and/or esophageal raft cells are contacted with a retinoic acid pathway activator. In some embodiments, the retinoic acid pathway activator comprises retinoic acid, all-trans retinoic acid, 9-cis retinoic acid, CD437, EC23, BS 493, TTNPB, or AM580, or any combination thereof. In some embodiments, the cells are not treated with a retinoic acid pathway activator. The retinoic acid pathway activator provided herein may be used in combination with any of the other growth factors, pathway activators, or pathway inhibitors provided herein.

In some embodiments, pluripotent stem cells, definitive endoderm cells, anterior foregut cells, dorsal anterior foregut cells, esophageal progenitor cells, and/or esophageal raft cells are contacted with an epidermal growth factor (EGF) pathway activator. In some embodiments, the EGF pathway activator comprises EGF, TGF-alpha, AR, BTC, HB-EGF, EPR, tomoregulin, NRG-1, NRG-2, NRG-3, or NRG-4, or any combination thereof. In some embodiments, the cells are not treated with an EGF pathway activator. The EGF pathway activator provided herein may be used in combination with any of the other growth factors, pathway activators, or pathway inhibitors provided herein.

In some embodiments, pluripotent stem cells, definitive endoderm cells, anterior foregut cells, dorsal anterior foregut cells, esophageal progenitor cells, and/or esophageal raft cells are contacted with a ROCK inhibitor (ROCKi). In some embodiments, the ROCKi comprises Y-27632, Y-30141, Y-39983, Ki-23095, SLx-2119, thiazovivin, azaindole 1, fasudil, ripasudil, netarsudil, RKI-1447, or GSK429286A, or any combination thereof. In some embodiments, the cells are not treated with a ROCKi. The ROCKi provided herein may be used in combination with any of the other growth factors, pathway activators, or pathway inhibitors provided herein.

In some embodiments, pluripotent stem cells, definitive endoderm cells, anterior foregut cells, dorsal anterior foregut cells, esophageal progenitor cells, and/or esophageal raft cells are contacted with a transforming growth factor-beta (TGF-beta) pathway activator or TGF-beta pathway inhibitor. In some embodiments, the TGF-beta family comprises bone morphogenetic protein (BMP), growth and differentiation factor (GDF), anti-Müllerian hormone, Activin, and Nodal pathways. In some embodiments, the TGF-beta pathway activator comprises TGF-beta 1, TGF-beta 2, TGF-beta 3, Activin A, Activin B, Nodal, a BMP, IDE1, IDE2, or any combination thereof. In some embodiments, the TGF-beta pathway inhibitor comprises A-83-01, DMH1, RepSox, LY365947, LY2109761, LY364947, SB431542, SB525334, SB505125, galunisertib, GW788388, LDN-193189, LDN-212854, hesperetin, or any combination thereof. In some embodiments, the cells are not treated with a TGF-beta pathway activator or TGF-beta pathway inhibitor. The TGF-beta pathway activator or TGF-beta pathway inhibitor provided herein may be used in combination with any of the other growth factors, pathway activators, or pathway inhibitors provided herein.

In some embodiments, pluripotent stem cells, definitive endoderm cells, anterior foregut cells, dorsal anterior foregut cells, esophageal progenitor cells, and/or esophageal raft cells are contacted with a SMAD pathway inhibitor. In some embodiments, the SMAD pathway inhibitor is a TGF-beta pathway inhibitor. In some embodiments, the SMAD pathway inhibitor comprises A-83-01, DMH1, RepSox, LY365947, LY2109761, LY364947, SB431542, SB525334, SB505125, galunisertib, GW788388, LDN-193189, LDN-212854, hesperetin, or any combination thereof. In some embodiments, the cells are not treated with a SMAD pathway inhibitor. The SMAD pathway inhibitor provided herein may be used in combination with any of the other growth factors, pathway activators, or pathway inhibitors provided herein.

In some embodiments, cells are differentiated via a “one step” process. For example, one or more molecules that can differentiate pluripotent stem cells into DE culture (e.g., Activin A) are combined with additional molecules that can promote directed differentiation of DE culture (e.g., FGF4, Wnt, Noggin, RA) to directly treat pluripotent stem cells.

In some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, pluripotent stem cells (e.g., embryonic stem cells or induced pluripotent stem cells) are expanded in cell culture. In some embodiments, pluripotent stem cells (e.g., iPSCs) are expanded in Matrigel. In some embodiments, the pluripotent stem cells are expanded in cell culture comprising a ROCK inhibitor (e.g. Y-27632). In some embodiments, the pluripotent stem cells (e.g., iPSCs) are differentiated into definitive endoderm cells. In the pluripotent stem cells (e.g., iPSCs) are differentiated into definitive endoderm cells by contacting the pluripotent stem cells (e.g., iPSCs) with Activin A, BMP4, or both. In some embodiments, the pluripotent stem cells (e.g. iPSCs) are contacted with a concentration of Activin A that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration of Activin A within a range defined by any two of the aforementioned concentrations, for example, 10 to 200 ng/mL, 10 to 100 ng/mL, 100 to 200 ng/mL, or 50 to 150 ng/mL. In some embodiments, the pluripotent stem cells (e.g., iPSCs) are contacted with a concentration of BMP4 that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, or 200 ng/mL, or any concentration of BMP4 within a range defined by any two of the aforementioned concentrations, for example, 1 to 200 ng/mL, 1 to 100 ng/mL, 25 to 200 ng/mL, 1 to 80 ng/mL, or 25 to 100 ng/mL. In some embodiments, the pluripotent stem cells are differentiated into definitive endoderm cells in a medium containing growth serum. In some embodiments, the medium contains 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% growth serum, or any percentage of growth serum within a range defined by any two of the aforementioned percentages, for example, 0-2%, 1-2.5%, 1.5-2.5%, 1.5-2%, or 0.5-2%. In some embodiments, the definitive endoderm cells are differentiated into anterior foregut cells in a medium containing FBS. In some embodiments, the medium contains 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% FBS, or any percentage of FBS within a range defined by any two of the aforementioned percentages, for example, 0-2%, 1-2.5%, 1.5-2.5%, 1.5-2%, or 0.5-2%. In some embodiments, the pluripotent stem cells are differentiated in growth media with stepwise increases in FBS concentration, for example, by exchanging media containing more than one of 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% FBS. In some embodiments, the pluripotent stem cells are differentiated in stepwise manner with media containing 0%, 0.2%, and 2% FBS. In addition to these embodiments, pluripotent stem cells (e.g., iPSCs) can be differentiated into definitive endoderm cells according to any other method known in the art.

In some embodiments, the definitive endoderm cells are differentiated into anterior foregut cells. In some embodiments, the anterior foregut cells are grown as a monolayer. In some embodiments, the definitive endoderm cells are differentiated into anterior foregut cells by contacting the definitive endoderm cells with one or more (e.g. at least 1, 2, 3, 4) of a Wnt protein or pathway activator, an FGF protein or pathway activator, a BMP pathway inhibitor, or a retinoic acid pathway activator, or any combination thereof. In some embodiments, the Wnt protein or pathway activator is Wnt3a. In some embodiments, the FGF protein or pathway activator is FGF4. In some embodiments, the BMP pathway inhibitor is Noggin. In some embodiments, the retinoic acid pathway activator is retinoic acid. In some embodiments, the Wnt protein or pathway activator, FGF protein or pathway activator, BMP pathway inhibitor, or retinoic acid pathway activator, or any combination thereof, are provided at concentrations that are, are about, are at least, are at least about, are not more than, or are not more than about, 0, 100, 120, 140, 160, 180, 200, 220, 240, 260, 280, 300, 320, 340, 360, 380, 400, 420, 440, 460, 480, 500, 520, 540, 560, 580, or 600 ng/mL, or any concentrations within a range defined by any two of the aforementioned concentrations, for example, 0 to 600 ng/mL, 0 to 200 ng/mL, 200 to 500 ng/mL, or 200 to 600 ng/mL. In some embodiments, the Wnt protein or pathway activator, FGF protein or pathway activator, BMP pathway inhibitor, or retinoic acid pathway activator, or any combination thereof, are provided at concentrations that are, are about, are at least, are at least about, are not more than, or are not more than about, 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 or any concentrations within a range defined by any two of the aforementioned concentrations, for example, 0 to 3.0 μM, 1.0 to 3.0 μM to 2.0 μM, or 1.5 to 3.0 μM. In some embodiments, the definitive endoderm cells are contacted with one or more (e.g. at least 1, 2, 3, 4) of the Wnt protein or pathway activator, FGF protein or pathway activator, BMP pathway inhibitor, or retinoic acid pathway activator, or any combination thereof, for a number of days is, is about, is at least, is at least about, is not more than, or is not more than about, 1, 2, 3, 4, 5, 6, 7, or 8 days. In some embodiments, the definitive endoderm cells are differentiated into anterior foregut cells without contacting the definitive endoderm cells with one or more (e.g. at least 1, 2, 3, 4) of a Wnt protein or pathway activator, FGF protein or pathway activator, BMP pathway inhibitor, or retinoic acid pathway activator, or any combination thereof. In some embodiments, the definitive endoderm cells are differentiated into anterior foregut cells without contacting the definitive endoderm cells with one or more (e.g. at least 1, 2, 3, 4) of Wnt3a, FGF4, Noggin, or retinoic acid, or any combination thereof. In some embodiments, the definitive endoderm cells are differentiated into anterior foregut cells in a medium containing growth serum. In some embodiments, the medium contains 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% growth serum, or any percentage of growth serum within a range defined by any two of the aforementioned percentages, for example, 0-2%, 1-2.5%, 1.5-2.5%, 1.5-2%, or 0.5-2%. In some embodiments, the definitive endoderm cells are differentiated into anterior foregut cells in a medium containing FBS. In some embodiments, the medium contains 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% FBS, or any percentage of FBS within a range defined by any two of the aforementioned percentages, for example, 0-2%, 1-2.5%, 1.5-2.5%, 1.5-2%, or 0.5-2%.

In addition to these embodiments, definitive endoderm cells can be differentiated into anterior foregut cells according to any other method known in the art.

### Formation of Dorsal Anterior Foregut Cells

Methods for producing anterior foregut cells from pluripotent stem cells disclosed herein or otherwise known in the art are applicable to the methods described herein. In some embodiments, methods known in the art to produce anterior foregut spheroids can be applied to producing anterior foregut cells. In some embodiments, the anterior foregut cells are obtained as a cell monolayer during differentiation by excluding spontaneously generated anterior foregut spheroids. In some embodiments, the anterior foregut cells are obtained by dissociating spontaneously generated anterior foregut spheroids into single cells and plating the dissociated single cells to a monolayer. In some embodiments, the anterior foregut cells are obtained both as a cell monolayer and by dissociating spontaneously generated anterior foregut spheroids into single cells.

In some embodiments, the anterior foregut cells are differentiated into dorsal anterior foregut cells, expressing SOX2, HNF113, or both. In some embodiments, the anterior foregut cells are differentiated into dorsal anterior foregut cells by contacting the anterior foregut cells with one or more (e.g. at least 1, 2, 3) of an epidermal growth factor (EGF) pathway activator, a BMP pathway inhibitor, or an FGF pathway activator, or any combination thereof. In some embodiments, the anterior foregut cells are further contacted with a growth supplement. In some embodiments, the anterior foregut cells are differentiated into dorsal anterior foregut cells by contacting the anterior foregut cells with one or more (e.g. at least 1, 2, 3, 4) of an EGF pathway activator, a BMP pathway inhibitor, an FGF pathway activator, or a growth supplement, or any combination thereof. In some embodiments, the anterior foregut cells are further contacted with a neuronal progenitor inhibitor (i.e., a compound that inhibits growth and/or differentiation of neuronal progenitor cells and neuronal cell types). In some embodiments, the anterior foregut cells are differentiated into dorsal anterior foregut cells by contacting the anterior foregut cells with one or more (e.g. at least 1, 2, 3, 4) of an EGF pathway activator, a BMP pathway inhibitor, an FGF pathway activator, or a neuronal progenitor inhibitor, or any combination thereof. In some embodiments, the EGF pathway activator comprises EGF, TGF-alpha, AR, BTC, HB-EGF, EPR, tomoregulin, NRG-1, NRG-2, NRG-3, or NRG-4, or any combination thereof. In some embodiments, the BMP pathway inhibitor comprises Noggin, RepSox, LY364947, LDN193189, SB431542, or any combination thereof. In some embodiments, the FGF pathway activator comprises FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23, or any combination thereof. In some embodiments, the growth supplement is a serum-free growth supplement, such as any one of those known in the art, optionally CultureOne supplement. In some embodiments, the neuronal progenitor inhibitor comprises CultureOne supplement or cytarabine. In some embodiments, the anterior foregut cells are contacted with EGF, Noggin, or FGF4, or any combination thereof, including all 3, to differentiate the anterior foregut cells to dorsal anterior foregut cells. In some embodiments, the anterior foregut cells are contacted with EGF, Noggin, FGF4, or a neuronal progenitor inhibitor, or any combination thereof, including all 4, to differentiate the anterior foregut cells to dorsal anterior foregut cells.

In some embodiments, the anterior foregut cells are contacted with an EGF pathway activator. In some embodiments, the EGF pathway activator is or comprises EGF. In some embodiments, the anterior foregut cells are contacted with the EGF pathway activator (e.g. EGF) at a concentration that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 10 to 200 ng/mL, 10 to 150 ng/mL, or 50 to 200 ng/mL. In some embodiments, the anterior foregut cells are contacted with the EGF pathway activator (e.g. EGF) at a concentration that is, is about, is at least, is at least about, is not more than, or is not more than about, 100 ng/mL.

In some embodiments, the anterior foregut cells are contacted with a BMP pathway inhibitor. In some embodiments, the BMP pathway inhibitor is or comprises Noggin. In some embodiments, the anterior foregut cells are contacted with the BMP pathway inhibitor (e.g. Noggin) at a concentration that is, is about, is at least, is at least about, is not more than, or is not more than about, 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations, for example, 100 to 300 ng/mL, 100 to 250 ng/mL, or 150 to 300 ng/mL. In some embodiments, the anterior foregut cells are contacted with the BMP pathway inhibitor (e.g. Noggin) at a concentration that is, is about, is at least, is at least about, is not more than, or is not more than about, 200 ng/mL.

In some embodiments, the anterior foregut cells are contacted with an FGF pathway activator. In some embodiments, the FGF pathway activator is or comprises FGF10. In some embodiments, the anterior foregut cells are contacted with the FGF pathway activator (e.g. FGF10) at a concentration that is, is about, is at least, is at least about, is not more than, or is not more than about, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 5 to 100 ng/mL, 5 to 75 ng/mL, or 25 to 100 ng/mL. In some embodiments, the anterior foregut cells are contacted with the FGF pathway activator (e.g. FGF10) at a concentration that is, is about, is at least, is at least about, is not more than, or is not more than about, 50 ng/mL.

In some embodiments, the anterior foregut cells are contacted with a growth supplement. In some embodiments, the growth supplement is a serum-free growth supplement, such as those generally known in the art. In some embodiments, the growth supplement is or comprises CultureOne supplement (GIBCO, Carlsbad, CA, USA). In some embodiments, the anterior foregut cells are contacted with the growth supplement (e.g. CultureOne) at a concentration of the growth supplement that is, is about, is at least, is at least about, is not more than, or is not more than about, 0.25×, 0.5×, 0.75×, 1×, 1.25×, 1.5×, 1.75×, or 2× according to the manufacturer's suggested concentration. In some embodiments, the anterior foregut cells are contacted with the growth supplement (e.g. CultureOne) at a concentration that is, is about, is at least, is at least about, is not more than, or is not more than about, 1×. In some embodiments, the CultureOne supplement or other growth supplement is used to inhibit the growth of neuronal progenitor cells during culture of the anterior foregut cells.

In some embodiments, the anterior foregut cells are contacted with a neuronal progenitor inhibitor. In some embodiments, the neuronal progenitor inhibitor is or comprises CultureOne supplement or cytarabine. In some embodiments, the anterior foregut cells are contacted with the neuronal progenitor inhibitor (e.g. CultureOne or cytarabine) at a concentration of the neuronal progenitor inhibitor that is, is about, is at least, is at least about, is not more than, or is not more than about, 0.25×, 0.5×, 0.75×, 1×, 1.25×, 1.5×, 1.75×, or 2× according to the manufacturer's suggested concentration. In some embodiments, the anterior foregut cells are contacted with the neuronal progenitor inhibitor (e.g. CultureOne or cytarabine) at a concentration that is, is about, is at least, is at least about, is not more than, or is not more than about, lx. In some embodiments, the CultureOne supplement, cytarabine, or other neuronal progenitor inhibitor is used to inhibit the growth of neuronal progenitor cells during culture of the pluripotent stem cells, definitive endoderm, and/or anterior foregut cells.

In some embodiments, the anterior foregut cells are contacted with one or more (e.g. at least 1, 2, 3, 4) of the EGF pathway activator, BMP pathway inhibitor, FGF pathway activator, or growth supplement for a number of days that is, is about, is at least, is at least about, is not more than, or is not more than about, 12, 13, 14, 15, 16, 17, 18, 19, 20, 21, 22, 23, or 24 hours, or 1, 2, 3, 4, 5, 6, 7, or 8 days, or a range defined by any two of the preceding values, for example, 12 hours—8 days, 1-8 days, or 2-6 days. In some embodiments, the anterior foregut cells are contacted with one or more (e.g. at least 1, 2, 3, 4) of the EGF pathway activator, BMP pathway inhibitor, FGF pathway activator, or neuronal progenitor inhibitor for a number of days that is, is about, is at least, is at least about, is not more than, or is not more than about, 12, 13, 14, 15, 16, 17, 18, 19, 20, 21, 22, 23, or 24 hours, or 1, 2, 3, 4, 5, 6, 7, or 8 days, or a range defined by any two of the preceding values, for example, 12 hours—8 days, 1-8 days, or 2-6 days.

### Formation of Esophageal Progenitor Cells

Methods for producing dorsal anterior foregut cells disclosed herein or otherwise known in the art are applicable to the methods disclosed herein.

In some embodiments, the dorsal anterior foregut cells are expanded and differentiated to esophageal progenitor cells. In some embodiments, the dorsal anterior foregut cells are dissociated into a single cell suspension. In some embodiments, the dorsal anterior foregut cells are dissociated using a dissociation enzyme. In some embodiments, the dissociation enzyme is or comprises one or more (e.g. at least 1, 2, 3, 4, 5) of trypsin, chymotrypsin, collagenase, elastase, or Accutase, or any combination thereof. In some embodiments, the single cell suspension of dorsal anterior foregut cells are plated onto a tissue culture container (e.g. tissue culture plate) or a portion thereof that is coated (notably the surfaces to which the cells are contacted) with an extracellular matrix or a component or mimetic thereof. In some embodiments, the extracellular matrix or component or mimetic thereof is allogeneic to the dorsal anterior foregut cells. In some embodiments, the dorsal anterior foregut cells are human and the extracellular matrix or component or mimetic thereof is human in origin. In some embodiments, the extracellular matrix or component or mimetic thereof is collagen type IV. In some embodiments, the collage type IV is human collagen type IV. In some embodiments, the collagen type IV is derived from human placenta. In some embodiments, the extracellular matrix or component or mimetic thereof does not comprise rat collagen type I matrix or Matrigel, or both.

In some embodiments, the dorsal anterior foregut cells are cultured in a growth medium in the tissue culture container. In some embodiments, the growth medium comprises an EGF pathway activator, bovine pituitary extract (BPE), or a ROCK inhibitor, or any combination thereof, including all three. In some embodiments, the dorsal anterior foregut cells are contacted with an EGF pathway activator, BPE, or a ROCK inhibitor, or any combination thereof, including all three. In some embodiments, the growth medium is a serum free medium. In some embodiments, the growth medium is Keratinocyte SFM (GIBCO, Carlsbad, CA, USA). In some embodiments, the growth medium comprises EGF, BPE, or a ROCK inhibitor, or any combination thereof. In some embodiments, the EGF pathway activator comprises EGF, TGF-alpha, AR, BTC, HB-EGF, EPR, tomoregulin, NRG-1, NRG-2, NRG-3, or NRG-4, or any combination thereof. In some embodiments, the ROCK inhibitor comprises Y-27632, Y-30141, Y-39983, Ki-23095, SLx-2119, thiazovivin, azaindole 1, fasudil, ripasudil, netarsudil, RKI-1447, or GSK429286A, or any combination thereof. In some embodiments, the growth medium comprises EGF, BPE, or Y-27632, or any combination thereof, including all three. In some embodiments, the dorsal anterior foregut cells are contacted with EGF, BPE, or Y-27632, or any combination thereof, including all three.

In some embodiments, the dorsal anterior foregut cells are contacted with an EGF pathway activator. In some embodiments, the EGF pathway activator is or comprises EGF. In some embodiments, the dorsal anterior foregut cells are contacted with the EGF pathway activator (e.g. EGF) at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 ng/mL, or any concentration within a range defined by any two of the aforementioned concentrations, for example, 1-10 ng/mL, 10-20 ng/mL, 5-15 ng/mL, or 8-12 ng/mL. In some embodiments, the dorsal anterior foregut cells are contacted with the EGF pathway activator (e.g. EGF) at a concentration that is, is about, is at least, is at least about, is not more than, or is not more than about, 10 ng/mL.

In some embodiments, the dorsal anterior foregut cells are contacted with BPE. In some embodiments, the dorsal anterior foregut cells are contacted with BPE at a concentration that is, is about, is at least, is at least about, is not more than, or is not more than about, 5, 10, 20, 30, 40, 50, 60, 70, 80, 90, or 100 μg/mL, or any concentration within a range defined by any two of the aforementioned concentrations, for example, 5-50 μg/mL, 20-100 μg/mL, 20-60 μg/mL, or 10-50 μg/mL. In some embodiments, the dorsal anterior foregut cells are contacted with BPE at a concentration that is, is about, is at least, is at least about, is not more than, or is not more than about, 30 μg/mL.

In some embodiments, the dorsal anterior foregut cells are contacted with a ROCK inhibitor. In some embodiments, the ROCK inhibitor is or comprises Y-27632. In some embodiments, the dorsal anterior foregut cells are contacted with the ROCK inhibitor (e.g. Y-27632) at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 15 μM, or 5 to 20 μM. In some embodiments, the dorsal anterior foregut cells are contacted with the ROCK inhibitor (e.g. Y-27632) at a concentration that is, is about, is at least, is at least about, is not more than, or is not more than about, 10 μM.

In some embodiments, the dorsal anterior foregut cells are cultured on the coated tissue culture container for a number of days that is, is about, is at least, is at least about, is not more than, or is not more than about, 1, 2, 3, 4, 5, 6, 7, or 8 days, or a range defined by any two of the preceding values, for example, 1-8, 2-6, 4-8, or 1-4 days, to differentiate to esophageal progenitor cells.

In some embodiments, the dorsal anterior foregut cells are expanded and differentiated to esophageal progenitor cells. In some embodiments, the dorsal anterior foregut cells are dissociated into a single cell suspension. In some embodiments, the single cell suspension of dorsal anterior foregut cells are plated onto a tissue culture container (e.g. tissue culture plate) or a portion thereof that is coated (notably the surfaces to which the cells are contacted) with an extracellular matrix or a component or mimetic thereof. In some embodiments, the dorsal anterior foregut cells are cultured in a growth medium in the tissue culture container. In some embodiments, the growth medium comprises an EGF pathway activator, bovine pituitary extract (BPE), or a ROCK inhibitor, or any combination thereof, including one, two, or all three. In some embodiments, the dorsal anterior foregut cells are contacted with an EGF pathway activator, BPE, or a ROCK inhibitor, or any combination thereof, including one, two, or all three. In some embodiments, the growth medium is a serum free medium. In some embodiments, the dorsal anterior foregut cells are contacted with EGF, BPE, or Y-27632, or any combination thereof, including one, two, or all three. In some embodiments, the dorsal anterior foregut cells are contacted with an EGF pathway activator. In some embodiments, the EGF pathway activator is or comprises EGF. In some embodiments, the dorsal anterior foregut cells are contacted with the EGF pathway activator (e.g. EGF) at a concentration that is, is about, is at least, is at least about, is not more than, or is not more than about, 10 ng/mL. In some embodiments, the dorsal anterior foregut cells are contacted with BPE. In some embodiments, the dorsal anterior foregut cells are contacted with BPE at a concentration that is, is about, is at least, is at least about, is not more than, or is not more than about, 30 μg/mL. In some embodiments, the dorsal anterior foregut cells are contacted with a ROCK inhibitor. In some embodiments, the ROCK inhibitor is or comprises Y-27632. In some embodiments, the dorsal anterior foregut cells are contacted with the ROCK inhibitor (e.g. Y-27632) at a concentration that is, is about, is at least, is at least about, is not more than, or is not more than about, 10 μM. In some embodiments, the dorsal anterior foregut cells are cultured on the coated tissue culture container for a number of days that is, is about, is at least, is at least about, is not more than, or is not more than about, 1, 2, 3, 4, 5, 6, 7, or 8 days, or a range defined by any two of the preceding values, for example, 1-8, 2-6, 4-8, or 1-4 days, to differentiate to esophageal progenitor cells.

In some embodiments, the resultant esophageal progenitor cells express SOX2, P63 or HNF1β, or any combination thereof. In some embodiments, the resultant esophageal progenitor cells express SOX2 at greater levels compared to dorsal anterior foregut cells.

### Formation of Esophageal Raft Cells

In some embodiments, the esophageal progenitor cells differentiated from dorsal anterior foregut cells and expanded on the coated tissue culture container are dissociated into a single cell suspension. In some embodiments, the expanded esophageal progenitor cells are dissociated using a dissociation enzyme. In some embodiments, the dissociation enzyme is or comprises one or more (e.g. at least 1, 2, 3, 4, 5) of trypsin, chymotrypsin, collagenase, elastase, or Accutase, or any combination thereof. In some embodiments, the single cell suspension of expanded esophageal progenitor cells are plated onto an insert member (e.g. a transwell or cell insert) or a portion thereof that is coated (notably the portions to which the cells are contacted, or the permeable surface of the insert member) with an extracellular matrix or a component or mimetic thereof. In some embodiments, the insert member comprises a surface that is permeable to growth medium but not cells. In some embodiments, the surface that is permeable of the insert member comprises a pore size that is, is about, is at least, is at least about, is not more than, or is not more than about, 0.1, 0.5, 1, 2, 3, 4, 5, 6, 7, 8, 9, or 10 μm, or any pore size within a range defined by any two of the aforementioned sizes, for example, 0.1-10 μm, 0.1-5 μm, 1-5 μm, 2-8 μm. In some embodiments, the surface that is permeable of the insert member comprises a pore size of 3 μm or about 3 μm. In some embodiments, the extracellular matrix or component or mimetic thereof is allogeneic to the esophageal progenitor cells. In some embodiments, the esophageal progenitor cells are human and the extracellular matrix or component or mimetic thereof is human in origin. In some embodiments, the extracellular matrix or component or mimetic thereof is collagen type IV. In some embodiments, the collage type IV is human collagen type IV. In some embodiments, the collagen type IV is derived from human placenta. In some embodiments, the extracellular matrix or component or mimetic thereof does not comprise rat collagen type I matrix or Matrigel, or both. In some embodiments, the expanded esophageal progenitor cells are cultured in a growth medium. In some embodiments, the growth medium is Advanced DMEM/F12. In some embodiments, the insert member is positioned within the tissue culture container.

In some embodiments, the growth medium contained within the insert member comprises one or more (e.g. 1, 2, 3) of an EGF pathway activator, a ROCK inhibitor, and a SMAD pathway inhibitor. In some embodiments, the EGF pathway activator comprises EGF, TGF-alpha, AR, BTC, HB-EGF, EPR, tomoregulin, NRG-1, NRG-2, NRG-3, or NRG-4, or any combination thereof. In some embodiments, the ROCK inhibitor comprises Y-27632, Y-30141, Y-39983, Ki-23095, SLx-2119, thiazovivin, azaindole 1, fasudil, ripasudil, netarsudil, RKI-1447, or GSK429286A, or any combination thereof. In some embodiments, the SMAD pathway inhibitor comprises A-83-01, DMH1, RepSox, LY365947, LY2109761, LY364947, SB431542, SB525334, SB505125, galunisertib, GW788388, LDN-193189, LDN-212854, hesperetin, or any combination thereof. In some embodiments, the growth medium contained within the insert member comprises EGF, Y-27632, A-83-01, or DMH1, or any combination thereof, including all four.

In some embodiments, the esophageal progenitor cells are contacted with an EGF pathway activator. In some embodiments, the EGF pathway activator is or comprises EGF. In some embodiments, the esophageal progenitor cells are contacted with the EGF pathway activator (e.g. EGF) at a concentration that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 10-200 ng/mL, 10-100 ng/mL, 100-200 ng/mL, 50-150 ng/mL, or 80-120 ng/mL. In some embodiments, the esophageal progenitor cells are contacted with the EGF pathway activator (e.g. EGF) at a concentration that is, is about, is at least, is at least about, is not more than, or is not more than about, 100 ng/mL.

In some embodiments, the esophageal progenitor cells are contacted with a ROCK inhibitor. In some embodiments, the ROCK inhibitor is or comprises Y-27632. In some embodiments, the esophageal progenitor cells are contacted with the ROCK inhibitor (e.g. Y-27632) at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1-20 μM, 1-10 μM, 10-20 μM, 5-15 μM, or 8-12 μM. In some embodiments, the esophageal progenitor cells are contacted with the ROCK inhibitor (e.g. Y-27632) at a concentration that is, is about, is at least, is at least about, is not more than, or is not more than about, 10 μM.

In some embodiments, the esophageal progenitor cells are contacted with a SMAD pathway inhibitor. In some embodiments, the SMAD pathway inhibitor is or comprises DMH1 and A-83-01. In some embodiments, the esophageal progenitor cells are contacted with the SMAD pathway inhibitor (e.g. DMH1 and A-83-01) at a concentration that is, is about, is at least, is at least about, is not more than, or is not more than about, 0.1, 0.5, 1, 2, 3, 4, 5, 6, 7, 8, 9, or 10 μM, or any concentration within a range defined by any two of the aforementioned concentrations, for example, 0.1-10 μM, 0.5-2 μM, 0.1-2 μM, or 0.5-5 μM. In some embodiments, the esophageal progenitor cells are contacted with the SMAD pathway inhibitor (e.g. DMH1 and A-83-01) at a concentration that is, is about, is at least, is at least about, is not more than, or is not more than about, 1 μM (e.g. 1 μM for each of DMH1 and A-83-01).

In some embodiments, the expanded esophageal progenitor cells are contacted with one or more (e.g. 1, 2, 3) of the EGF pathway activator, ROCK inhibitor, and SMAD pathway inhibitor in the insert member. In some embodiments, the tissue culture container comprises EGF. In some embodiments, the expanded esophageal progenitor cells are cultured in the insert member for a number of days that is, is about, is at least, is at least about, is not more than, or is not more than about, 1, 2, 3, 4, 5, 6, 7, or 8 days, or a range defined by any two of the preceding values, for example, 1-8, 2-6, 4-8, or 1-4 days.

In some embodiments, the expanded esophageal progenitor cells are differentiated into an esophageal raft culture. In some embodiments, after contacting the expanded esophageal progenitor cells with one or more of the EGF, ROCK inhibitor, and SMAD inhibitor in the insert member, the growth medium in the insert member is removed and the tissue culture container contains an amount of growth medium such that the esophageal raft culture is only partially submerged in the growth medium, such that the esophageal raft culture is in an air-liquid interface. In some embodiments, the esophageal raft culture is cultured in an air-liquid interface. In some embodiments, the tissue culture container comprises EGF. In some embodiments, the esophageal raft culture is cultured in the air-liquid interface for a number of days that is, is about, is at least, is at least about, is not more than, or is not more than about, 5, 6, 7, 8, 9, 10, 11, 12, 13, 14, 15, 16, 17, 18, 19, 20, 21, 22, 23, 24, 25, 26, 27, 28, 29, or 30 days or a range defined by any two of the preceding values, for example, 5-30 days, 5-20 days, 10-25 days, 10-30 days, or 20-30 days. In some embodiments, culturing the esophageal raft culture in the air-liquid interface matures the esophageal raft culture.

In some embodiments, the esophageal progenitor cells differentiated from dorsal anterior foregut cells and expanded on the coated tissue culture container are dissociated into a single cell suspension. In some embodiments, the single cell suspension of expanded esophageal progenitor cells are plated onto an insert member (e.g. a transwell or cell insert) or a portion thereof that is coated (notably the portions to which the cells are contacted, or the permeable surface of the insert member) with an extracellular matrix or a component or mimetic thereof. In some embodiments, the insert member comprises a surface that is permeable to growth medium but not cells. In some embodiments, the surface that is permeable of the insert member comprises a pore size that is, is about, is at least, is at least about, is not more than, or is not more than about, 0.1, 0.5, 1, 2, 3, 4, 5, 6, 7, 8, 9, or 10 μm, or any pore size within a range defined by any two of the aforementioned sizes, for example, 0.1-10 μm, 0.1-5 μm, 1-5 μm, 2-8 μm. In some embodiments, the surface that is permeable of the insert member comprises a pore size of 3 μm or about 3 μm. In some embodiments, the insert member is positioned within the tissue culture container. In some embodiments, the growth medium contained within the insert member comprises one or more (e.g. 1, 2, 3) of an EGF pathway activator, a ROCK inhibitor, and a SMAD pathway inhibitor. In some embodiments, the esophageal progenitor cells are contacted with an EGF pathway activator. In some embodiments, the EGF pathway activator is or comprises EGF. In some embodiments, the esophageal progenitor cells are contacted with the EGF pathway activator (e.g. EGF) at a concentration that is, is about, is at least, is at least about, is not more than, or is not more than about, 100 ng/mL. In some embodiments, the esophageal progenitor cells are contacted with a ROCK inhibitor. In some embodiments, the ROCK inhibitor is or comprises Y-27632. In some embodiments, the esophageal progenitor cells are contacted with the ROCK inhibitor (e.g. Y-27632) at a concentration that is, is about, is at least, is at least about, is not more than, or is not more than about, 10 μM. In some embodiments, the esophageal progenitor cells are contacted with a SMAD pathway inhibitor. In some embodiments, the SMAD pathway inhibitor is or comprises DMH1 and A-83-01. In some embodiments, the esophageal progenitor cells are contacted with the SMAD pathway inhibitor (e.g. DMH1 and A-83-01) at a concentration that is, is about, is at least, is at least about, is not more than, or is not more than about, 1 μM (e.g. 1 μM for each of DMH1 and A-83-01). In some embodiments, the expanded esophageal progenitor cells are contacted with one or more (e.g. 1, 2, 3) of the EGF pathway activator, ROCK inhibitor, and SMAD pathway inhibitor in the insert member. In some embodiments, the tissue culture container comprises EGF. In some embodiments, the expanded esophageal progenitor cells are cultured in the insert member for a number of days that is, is about, is at least, is at least about, is not more than, or is not more than about, 1, 2, 3, 4, 5, 6, 7, or 8 days, or a range defined by any two of the preceding values, for example, 1-8, 2-6, 4-8, or 1-4 days. In some embodiments, the expanded esophageal progenitor cells are differentiated into an esophageal raft culture. In some embodiments, after contacting the expanded esophageal progenitor cells with one or more of the EGF, ROCK inhibitor, and SMAD inhibitor in the insert member, the growth medium in the insert member is removed and the tissue culture container contains an amount of growth medium such that the esophageal raft culture is only partially submerged in the growth medium, such that the esophageal raft culture is in an air-liquid interface. In some embodiments, the esophageal raft culture is cultured in an air-liquid interface. In some embodiments, the tissue culture container comprises EGF. In some embodiments, the esophageal raft culture is cultured in the air-liquid interface for a number of days that is, is about, is at least, is at least about, is not more than, or is not more than about, 5, 6, 7, 8, 9, 10, 11, 12, 13, 14, 15, 16, 17, 18, 19, 20, 21, 22, 23, 24, 25, 26, 27, 28, 29, or 30 days or a range defined by any two of the preceding values, for example, 5-30 days, 5-20 days, 10-25 days, 10-30 days, or 20-30 days. In some embodiments, culturing the esophageal raft culture in the air-liquid interface matures the esophageal raft culture.

In some embodiments, the dissociated esophageal progenitor cells are combined with enteric neural crest cells (ENCCs) and the combined esophageal progenitor cells and ENCCs are cultured to form an innervated esophageal raft culture. In some embodiments, the innervated esophageal raft culture comprises enteric neural crest cells (ENCCs), neuronal progenitor cells and/or βIII-tubulin+ neuronal cells. In some embodiments, the neuronal progenitor cells are SOX10+. In some embodiments, the esophageal progenitor cells and ENCCs are combined by low-speed centrifugation, or other method of aggregating cells without excessive disruption. In some embodiments, the ENCCs are isolated as single cells derived from neurospheres, which can be derived from pluripotent stem cells. Methods for producing ENCCs are generally known in the art, and methods for combining them with organoids are explored in WO 2016/061464, which is hereby expressly incorporated by reference in its entirety.

### Exemplary Methods for Esophageal Raft Culture

In some embodiments, the esophageal raft culture is prepared from anterior foregut cells. In some embodiments, the esophageal raft culture and anterior foregut cells are prepared originally from iPSCs. In some embodiments, the iPSCs are hiPSCs. In some embodiments, the anterior foregut cells are differentiated from iPSCs according to one or more of the methods disclosed herein. In some embodiments, the methods comprise culturing iPSCs under conditions to differentiate the iPSCs into definitive endoderm cells, culturing the definitive endoderm cells under conditions to differentiate the definitive endoderm cells into anterior foregut cells. In some embodiments, the methods comprise culturing iPSCs with a TGF-beta superfamily growth factor to differentiate the iPSCs into definitive endoderm cells, culturing the definitive endoderm cells with one or more (e.g. at least 1, 2, 3, 4) of a Wnt protein or pathway activator, an FGF protein or activator, a BMP pathway inhibitor, or a retinoic acid pathway activator to differentiate the definitive endoderm cells into anterior foregut cells. In some embodiments, the methods comprise culturing iPSCs with Activin A, or BMP4, or both to differentiate the iPSCs into definitive endoderm cells, culturing the definitive endoderm cells with one or more (e.g. at least 1, 2, 3, 4) of Wnt3a, FGF4, Noggin, or retinoic acid to differentiate the definitive endoderm cells into anterior foregut cells. In some embodiments, the iPSCs are cultured with a concentration of Activin A that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration of Activin A within a range defined by any two of the aforementioned concentrations, for example, 10 to 200 ng/mL, 10 to 100 ng/mL, 100 to 200 ng/mL, or 50 to 150 ng/mL. In some embodiments, the iPSCs are cultured with 100 ng/mL Activin A. In some embodiments, the iPSCs are cultured with a concentration of BMP4 that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, or 200 ng/mL, or any concentration of BMP4 within a range defined by any two of the aforementioned concentrations, for example, 1 to 200 ng/mL, 1 to 100 ng/mL, 25 to 200 ng/mL, 1 to 80 ng/mL, or 25 to 100 ng/mL. In some embodiments, the iPSCs are cultured with 50 ng/mL BMP4. In some embodiments, the iPSCs are cultured with Activin A, or BMP4, or both for 1, 2, 3, 4, or 5 days. In some embodiments, the definitive endoderm cells are cultured with one or more (e.g. at least 1, 2, 3, 4) of Wnt3a, FGF4, Noggin, or retinoic acid, or any combination thereof, at a concentration of each sufficient to differentiate the definitive endoderm cells to anterior foregut cells. In some embodiments, the definitive endoderm cells are cultured with one or more (e.g. at least 1, 2, 3, 4) of Wnt3a, FGF4, Noggin, or retinoic acid, or any combination thereof, at concentrations that are, are about, are at least, are at least about, are not more than, or are not more than about, 0, 100, 120, 140, 160, 180, 200, 220, 240, 260, 280, 300, 320, 340, 360, 380, 400, 420, 440, 460, 480, 500, 520, 540, 560, 580, or 600 ng/mL, or any concentrations within a range defined by any two of the aforementioned concentrations, for example, 0 to 600 ng/mL, 0 to 200 ng/mL, 200 to 500 ng/mL, or 200 to 600 ng/mL. In some embodiments, the definitive endoderm cells are cultured with one or more (e.g. at least 1, 2, 3, 4) of Wnt3a, FGF4, Noggin, or retinoic acid, or any combination thereof, at concentrations that are, are about, are at least, are at least about, are not more than, or are not more than about, 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 μM, or any concentrations within a range defined by any two of the aforementioned concentrations, for example, 0 to 3.0 μM, 1.0 to 3.0 μM, 0 to 2.0 μM, or 1.5 to 3.0 μM. In some embodiments, the definitive endoderm cells are cultured with 500 ng/mL or about 500 ng/mL Wnt3a, 500 ng/mL or about 500 ng/mL FGF4, 200 ng/mL or about 200 ng/mL Noggin, and 2 μM or about 2 μM retinoic acid. In some embodiments, the definitive endoderm cells are cultured with one or more (e.g. at least 1, 2, 3, 4) of Wnt3a, FGF4, Noggin, or retinoic acid for 1, 2, 3, 4, or 5 days.

In some embodiments, the esophageal raft culture is prepared from anterior foregut cells produced by one or more of the methods disclosed herein. In some embodiments, the esophageal raft culture is prepared by culturing the anterior foregut cells under conditions to differentiate the anterior foregut cells into dorsal anterior foregut cells, culturing the dorsal anterior foregut cells under conditions to differentiate the dorsal anterior foregut cells to esophageal progenitor cells, and culturing the esophageal progenitor cells under conditions to differentiate the esophageal progenitor cells into an esophageal raft culture. In some embodiments, the anterior foregut cells are cultured as a monolayer. In some embodiments, the anterior foregut cells are not cultured as spheroids. In some embodiments, the esophageal progenitor cells are cultured to expand the esophageal progenitor cells before culturing the esophageal progenitor cells under conditions to differentiate the esophageal progenitor cells into the esophageal raft culture. In some embodiments, the conditions to differentiate the esophageal progenitor cells into the esophageal raft culture comprises culturing the esophageal progenitor cells in an air-liquid interface.

In some embodiments, the esophageal raft culture is prepared from anterior foregut cells produced by one or more of the methods disclosed herein. In some embodiments, the esophageal raft culture is prepared by culturing the anterior foregut cells with one or more (e.g. at least 1, 2, 3, 4) of an EGF pathway activator, a BMP pathway inhibitor, an FGF pathway activator, or a growth supplement, or by culturing the anterior foregut cells with one or more (e.g. at least 1, 2, 3, 4) of an EGF pathway activator, a BMP pathway inhibitor, or an FGF pathway activator, optionally a neural progenitor inhibitor, or any combination thereof, to differentiate the anterior foregut cells into dorsal anterior foregut cells, dissociating the dorsal anterior foregut cells into single cells and culturing the dorsal anterior foregut cells in a first tissue culture container comprising a ROCK inhibitor to differentiate the dorsal anterior foregut cells to esophageal progenitor cells, dissociating the esophageal progenitor cells into single cells and culturing the esophageal progenitor cells in, and/or on a surface of, an insert member (e.g. transwell) positioned within a second tissue culture container, where the insert member comprises a surface that is permeable to a growth medium but not cells and where the insert member and second tissue culture container comprise an amount of growth medium such that the esophageal progenitor cells are fully submerged in growth medium, and culturing the esophageal progenitor cells in an air-liquid interface to differentiate the esophageal progenitor cells into an esophageal raft culture. In some embodiments, the second tissue culture container is the same as the first tissue culture container. In some embodiments, the anterior foregut cells are contacted with EGF, Noggin, and FGF10. In some embodiments, the anterior foregut cells are contacted with EGF, Noggin, FGF10, and CultureOne supplement or some other neuronal progenitor inhibitor (e.g., cytarabine). In some embodiments, the anterior foregut cells are contacted with the EGF pathway activator (e.g. EGF) at a concentration that that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 10 to 200 ng/mL, 10 to 150 ng/mL, or 50 to 200 ng/mL. In some embodiments, the anterior foregut cells are contacted with the BMP pathway inhibitor (e.g. Noggin) at a concentration that is, is about, is at least, is at least about, is not more than, or is not more than about, 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations, for example, 100 to 300 ng/mL, 100 to 250 ng/mL, or 150 to 300 ng/mL. In some embodiments, the anterior foregut cells are contacted with the FGF pathway activator (e.g. FGF10) at a concentration that is, is about, is at least, is at least about, is not more than, or is not more than about, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 5 to 100 ng/mL, 5 to 75 ng/mL, or 25 to 100 ng/mL. In some embodiments, the anterior foregut cells are contacted with the growth supplement (e.g. CultureOne) or another neuronal progenitor inhibitor (e.g., cytarabine) at a concentration supplement that is, is about, is at least, is at least about, is not more than, or is not more than about, 0.25×, 0.5×, 0.75×, 1×, 1.25×, 1.5×, 1.75×, or 2× according to the manufacturer's suggested concentration. In some embodiments, the growth supplement or neuronal progenitor inhibitor is provided at 1×. In some embodiments, the anterior foregut cells are cultured as a monolayer. In some embodiments, the anterior foregut cells are not cultured as spheroids. In some embodiments, the dorsal anterior foregut cells are cultured in the first tissue culture container on extracellular matrix or a component or mimetic thereof. In some embodiments, the esophageal progenitor cells are cultured in, and/or on a surface of, the insert member on extracellular matrix or a component or mimetic thereof. In some embodiments, the esophageal progenitor cells are cultured in the insert member with one or more (e.g. at least 1, 2, 3) of an EGF pathway activator, a ROCK inhibitor, or a SMAD inhibitor, or any combination thereof, and with EGF in the second tissue culture container. In some embodiments, the air-liquid interface comprises removing the growth medium from the insert member such that the second tissue culture container and/or insert member contains an amount of growth medium such that the esophageal progenitor cells are only partially submerged in the growth medium. In some embodiments, the esophageal progenitor cells are contacted with EGF, Y-27632, DMH1, and A-83-01. In some embodiments, the esophageal progenitor cells are contacted with the EGF pathway activator (e.g. EGF) at a concentration that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 10-200 ng/mL, 10-100 ng/mL, 100-200 ng/mL, 50-150 ng/mL, or 80-120 ng/mL. In some embodiments, the esophageal progenitor cells are contacted with the ROCK inhibitor (e.g. Y-27632) at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1-20 μM, 1-10 μM, 10-20 μM, 5-15 μM, or 8-12 μM. In some embodiments, the esophageal progenitor cells are contacted with the SMAD inhibitor (e.g. DMH1 and A-83-01) at a concentration at a concentration that is, is about, is at least, is at least about, is not more than, or is not more than about, 0.1, 0.5, 1, 2, 3, 4, 5, 6, 7, 8, 9, or 10 μM, or any concentration within a range defined by any two of the aforementioned concentrations for example, 0.1-10 μM, 0.5-2 μM, 0.1-2 μM, or 0.5-5 μM (e.g. for each of DMH1 and A-83-01).

In some embodiments, the esophageal raft culture is prepared from anterior foregut cells produced by one or more of the methods disclosed herein. In some embodiments, the esophageal raft culture is prepared by culturing the anterior foregut cells with one or more (e.g. at least 1, 2, 3, 4) of EGF, Noggin, FGF10, or CultureOne supplement to differentiate the anterior foregut cells into dorsal anterior foregut cells, dissociating the dorsal anterior foregut cells into single cells and culturing the dorsal anterior foregut cells in a first tissue culture container with Y-27632 to differentiate the dorsal anterior foregut cells to esophageal progenitor cells, dissociating the esophageal progenitor cells into single cells and culturing the esophageal progenitor cells in, and/or on a surface of, an insert member (e.g. transwell) positioned within a second tissue culture container, where the insert member comprises a surface that is permeable to the growth medium but not cells and where the insert member and second tissue culture container comprise an amount of growth medium such that the esophageal progenitor cells are fully submerged in growth medium, and culturing the esophageal progenitor cells in an air-liquid interface to differentiate the esophageal progenitor cells into an esophageal raft culture. In some embodiments, the second tissue culture container is the same as the first tissue culture container. In some embodiments, the anterior foregut cells are cultured as a monolayer. In some embodiments, the anterior foregut cells are not cultured as spheroids. In some embodiments, the dorsal anterior foregut cells are cultured in the first tissue culture container on collagen type IV. In some embodiments, the esophageal progenitor cells are cultured in, and/or on a surface of, the insert member on collagen type IV. In some embodiments, the esophageal progenitor cells are cultured in, and/or on a surface of, the insert member with one or more (e.g. at least 1, 2, 3, 4) of EGF, Y-27632, DMIH1 or A-83-01 and with EGF in the second tissue culture container. In some embodiments, the air-liquid interface comprises removing the growth medium from the insert member such that the second tissue culture container and/or insert member contains an amount of growth medium such that the esophageal progenitor cells are only partially submerged in the growth medium. In some embodiments, the anterior foregut cells are cultured with one or more (e.g. at least 1, 2, 3) of EGF, Noggin, or FGF10 at concentrations that are, are about, are at least, are at least about, are not more than, or are not more than about, 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 ng/mL, or any concentrations within a range defined by any two of the aforementioned concentrations, for example, 10 to 300 ng/mL, 10 to 200 ng/mL, 100 to 200 ng/mL, or 50 to 200 ng/mL. In some embodiments, the anterior foregut cells are cultured with 100 ng/mL or about 100 ng/mL EGF. In some embodiments, the anterior foregut cells are cultured with 200 ng/mL or about 200 ng/mL Noggin. In some embodiments, the anterior foregut cells are cultured with 50 ng/mL or about 50 ng/mL FGF10. In some embodiments, the anterior foregut cells are cultured with 1× CultureOne supplement. In some embodiments, the anterior foregut cells are cultured for 1, 2, 3, 4 or 5 days. In some embodiments, the dorsal anterior foregut cells are dissociated with Accutase. In some embodiments, the dorsal anterior foregut cells are cultured in the first tissue culture container with Y-27632 at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentrations within a range defined by any two of the aforementioned concentrations, for example, 1 to 20 μM, 5 to 15 μM, or 8 to 12 μM. In some embodiments, the dorsal anterior foregut cells are cultured in the first tissue culture container with 10 μM or about 10 μM Y-27632. In some embodiments, the dorsal anterior foregut cells are cultured in the first tissue culture container on 1.5 μg collagen type IV/cm² of culture surface area. In some embodiments, the esophageal progenitor cells are cultured in, and/or on a surface of, an insert member comprising a surface that is permeable to the growth medium but not cells. In some embodiments, the surface that is permeable of the insert member comprises a pore size that is, is about, is at least, is at least about, is not more than, or is not more than about, 0.1, 0.5, 1, 2, 3, 4, 5, 6, 7, 8, 9, or 10 μm, or any pore size within a range defined by any two of the aforementioned sizes, for example, 0.1 to 10 μm, 0.1 to 5 μm, 5 to 10 μm, or 1 to 5 μm. In some embodiments, the surface that is permeable of the insert member comprises a pore size that is, is about, is at least, is at least about, is not more than, or is not more than about, 3 μm. In some embodiments, the esophageal progenitor cells are cultured in, and/or on a surface of, the insert member with one or more (e.g. at least 1, 2, 3, 4) of EGF, Y-27632, DMH1, or A-83-01 at concentrations that are, are about, are at least, are at least about, are not more than, or are not more than about, 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 ng/mL, or any concentrations within a range defined by any two of the aforementioned concentrations, for example, 10 to 300 ng/mL, 10 to 200 ng/mL, 100 to 200 ng/mL, or 50 to 200 ng/mL. In some embodiments, the esophageal progenitor cells are cultured in, and/or on a surface of, the insert member with one or more (e.g. at least 1, 2, 3, 4) of EGF, Y-27632, DMH1, or A-83-01 at concentrations that are, are about, are at least, are at least about, are not more than, or are not more than about, 0.1, 0.5, 1, 2, 3, 4, 5, 6, 7, 8, 9, 10, 11, 12, 13, 14, 15, 16, 17, 18, 19, or 20 μM, or any concentrations within a range defined by any two of the aforementioned concentrations, for example, 0.1 to 20 μM, 5 to 15 μM, 0.1 to 4 μM, or 8 to 12 μM. In some embodiments, the esophageal progenitor cells are cultured in, and/or on a surface of, the insert member with 100 ng/mL or about 100 ng/mL EGF. In some embodiments, the esophageal progenitor cells are cultured in, and/or on a surface of, the insert member with 10 μM or about 10 μM Y-27632. In some embodiments, the esophageal progenitor cells are cultured in, and/or on a surface of, the insert member with 1 μM or about 1 μM DMH1. In some embodiments, the esophageal progenitor cells are cultured in, and/or on a surface of, the insert member with 1 μM or about 1 μM A-83-01. In some embodiments, the esophageal progenitor cells are cultured in, and/or on a surface of, the insert member with one or more (e.g. at least 1, 2, 3, 4) of EGF, Y-27632, DMH1, or A-83-01 for 1, 2, 3, 4, 5, 6, 7, or 8 days. In some embodiments, the esophageal progenitor cells are cultured in, and/or on a surface of, the insert member on 1.5 μg collagen type IV/cm′ of culture surface area. In some embodiments, the esophageal progenitor cells are cultured in the air-liquid interface with 100 ng/mL or about 100 ng/mL EGF in the second tissue culture container. In some embodiments, the esophageal progenitor cells are cultured in the air-liquid interface for 5, 6, 7, 8, 9, 10, 11, 12, 13, 14, 15, 16, 17, 18, 19, 20, 21, 22, 23, 24, 25, 26, 27, 28, 29, or 30 days.

In some embodiments, the esophageal raft culture is produced by one or more of the methods described herein. In some embodiments, the esophageal raft culture is a human esophageal raft culture. In some embodiments, the esophageal raft culture is derived from human cells. In some embodiments, the esophageal raft culture is derived from human iPSCs. In some embodiments, the esophageal raft culture is not derived from a spheroid or organoid. In some embodiments, the esophageal raft culture is not cultured on an extracellular matrix or component or mimetic thereof. In some embodiments, the esophageal raft culture is not cultured on rat collagen type I matrix or Matrigel, or both. In some embodiments, the esophageal raft culture is not cultured with xenogeneic components. In some embodiments, the esophageal raft culture is cultured on human collagen type IV. In some embodiments, the esophageal raft culture is not cultured on a feeder cell substrate. In some embodiments, the esophageal raft culture is not cultured on mouse fibroblasts. In some embodiments, the esophageal raft culture is not cultured on irradiated mouse fibroblasts.

### Properties of Esophageal Raft Cultures

In some embodiments, the esophageal raft culture is produced by one or more of the methods described herein. In some embodiments, the esophageal raft culture comprises a stratified squamous epithelium layer. In some embodiments, the stratified squamous epithelium layer comprises a suprabasal layer and a basal layer. In some embodiments, the stratified squamous epithelium layer is positive for E-cadherin (Ecad). In some embodiments, the suprabasal layer is positive for keratin 13 (KRT13), or keratin 8 (KRT8), or both. In some embodiments, the basal layer is positive for one or more (e.g. at least 1, 2, 3) of sex determining region Y-box 2 (SOX2), tumor protein p63 (P63), or keratin 5 (KRT5), or any combination thereof. In some embodiments, the esophageal raft culture comprises a mesenchyme layer. In some embodiments, the mesenchyme layer comprises muscle fibers. In some embodiments, the mesenchyme layer is positive for one or more (e.g. at least 1, 2, 3) of forkhead box protein F1 (FOXF1), homeobox protein Nkx-6.1 (NKX6-1), or vimentin, or any combination thereof. In some embodiments, the muscle fibers are positive for desmin. In some embodiments, the esophageal raft culture lacks a lamina propria layer, or has a reduced or substantially reduced lamina propria layer as compared to the esophageal tissue of an adult animal of the same species as the esophageal raft culture (e.g., reduced by 50%, 60%, 70%, 80%, 90% 95% or more). In some embodiments, the esophageal raft culture is effectively free of neuronal progenitor cells and/or βIII-tubulin+ neuronal cells. In some embodiments, the esophageal raft culture further comprises enteric neural crest cells (ENCCs). In some embodiments, the esophageal raft culture further comprises ENCCs, neuronal progenitor cells, and/or βIII-tubulin+ neuronal cells, such that the esophageal raft culture is an innervated raft culture. In some embodiments, the neuronal progenitor cells are SOX10+.

In some embodiments, the esophageal raft culture produced by any of the methods disclosed herein has a thickness that is, is about, is at least, is at least about, is not more than, or is not more than about, 1, 5, 10, 20, 30, 40, 50, 60, 70, 80, 90, 100, 200, 300, 400, or 500 μm, or any thickness within a range defined by any two of the aforementioned lengths, for example, 1 to 500 μm, 10 to 300 μm, 50 to 100 μm, 1 to 100 μm, or 100 to 500 μm. In some embodiments, the esophageal raft culture produced by any of the methods disclosed herein has a thickness that is, is about, is at least, is at least about, is not more than, or is not more than about, 150, 200, 250, 300, 350, 400, 450, or 500 μm, or any thickness within a range defined by any two of the aforementioned thicknesses, for example, 150 to 500 μm, 150 to 350 μm, or 250 to 500 μm.

In some embodiments, the esophageal raft culture produced by any of the methods disclosed herein has a surface area that is, is about, is at least, is at least about, is not more than, or is not more than about, 0.1, 0.5, 1, 5, 10, 15, 20, 25, 30, 40, 50, 60, 70, 80, 90, or 100 cm², or any surface area within a range defined by any two of the aforementioned surface areas, for example, 0.1 to 100 cm², 10 to 80 cm², 20 to 40 cm², 0.1 to 30 cm², or 50 to 100 cm². In some embodiments, the esophageal raft culture produced by any of the methods disclosed herein has a surface area that is, is about, is at least, is at least about, is not more than, or is not more than about, 0.1, 0.5, 1, 1.5, or 2 cm², or any surface area within a range defined by any two of the aforementioned surface areas, for example, 0.1 to 2 cm², 0.1 to 1 cm², or 0.5 to 2 cm².

In some embodiments, the esophageal raft culture produced by any of the methods disclosed herein has a volume that is, is about, is at least, is at least about, is not more than, or is not more than about, 10⁻⁵, 10⁻⁴, 10⁻³, 10⁻², 10⁻¹, 1, 5 or 10 cm³, or any volume within a range defined by any two of the aforementioned volumes, for example, 10⁻⁵ to 10 cm³, 10⁻² to 1 cm³, or 1 to 10 cm³.

In some embodiments, the stratified squamous epithelium layer of an esophageal raft culture produced by any of the methods disclosed herein has a thickness that is, is about, is at least, is at least about, is not more than, or is not more than about, 1, 5, 10, 20, 30, 40, 50, 60, 70, 80, 90, 100, 200, 300, 400, or 500 μm, or any thickness within a range defined by any two of the aforementioned lengths, for example, 1 to 500 μm, 20 to 200 μm, 50 to 100 μm, 1 to 100 μm, or 100 to 500 μm. In some embodiments, the stratified squamous epithelium layer of an esophageal raft culture produced by any of the methods disclosed herein has a thickness that is, is about, is at least, is at least about, is not more than, or is not more than about, 50, 60, 70, 80, 90, 100, 110, 120, 130, 140, 150, 160, 170, 180, 190, 200, 210, 220, 230, 240, or 250 μm, or any thickness within a range defined by any two of the aforementioned thicknesses, for example, 50 to 250 μm, 50 to 150 μm, or 100 to 250 μm.

In some embodiments, the suprabasal layer of an esophageal raft culture produced by any of the methods disclosed herein has a thickness that is, is about, is at least, is at least about, is not more than, or is not more than about, 1, 5, 10, 20, 30, 40, 50, 60, 70, 80, 90, 100, 200, 300, 400, or 500 μm, or any thickness within a range defined by any two of the aforementioned lengths, for example, 1 to 500 μm, 20 to 200 μm, 50 to 100 μm, 1 to 100 μm, or 100 to 500 μm. In some embodiments, the suprabasal layer of an esophageal raft culture produced by any of the methods disclosed herein has a thickness that is, is about, is at least, is at least about, is not more than, or is not more than about, 80, 90, 100, 110, 120, 130, 140, 150, 160, 170, 190, 190, or 200 μm, or any thickness within a range defined by any two of the aforementioned thicknesses, for example, 80 to 200 μm, 80 to 150 μm, or 100 to 200 μm.

In some embodiments, the basal layer of an esophageal raft culture produced by any of the methods disclosed herein has a thickness that is, is about, is at least, is at least about, is not more than, or is not more than about, 1, 5, 10, 20, 30, 40, 50, 60, 70, 80, 90, 100, 200, 300, 400, or 500 μm, or any thickness within a range defined by any two of the aforementioned lengths, for example, 1 to 500 μm, 20 to 200 μm, 50 to 100 μm, 1 to 100 μm, or 100 to 500 μm. In some embodiments, the basal layer of an esophageal raft culture produced by any of the methods disclosed herein has a thickness that is, is about, is at least, is at least about, is not more than, or is not more than about, 10, 20, 30, 40, 50, 60, 70, 80, 90, or 100 μm, or any thickness within a range defined by any two of the aforementioned thicknesses, for example, 10 to 100 μm, 10 to 50 μm, or 50 to 100 μm.

In some embodiments, the mesenchyme layer of an esophageal raft culture produced by any of the methods disclosed herein has a thickness that is, is about, is at least, is at least about, is not more than, or is not more than about, 1, 5, 10, 20, 30, 40, 50, 60, 70, 80, 90, 100, 200, 300, 400, or 500 μm, or any thickness within a range defined by any two of the aforementioned lengths, for example, 1 to 500 μm, 20 to 200 μm, 50 to 100 μm, 1 to 100 μm, or 100 to 500 μm. In some embodiments, the mesenchyme layer of an esophageal raft culture produced by any of the methods disclosed herein has a thickness that is, is about, is at least, is at least about, is not more than, or is not more than about, 100, 150, 200, 250, 300, 350, or 400 μm, or any thickness within a range defined by any two of the aforementioned thicknesses, for example, 100 to 400 μm, 100 to 200 μm, or 200 to 400 μm.

In some embodiments, the esophageal raft culture is produced by one or more of the methods described herein. In some embodiments, the esophageal raft culture comprises neuronal structures. In some embodiments, the esophageal raft culture comprises cells that express neuronal markers, such as SOX2 or βIII-tubulin.

In some embodiments, the esophageal raft culture does not comprise vascularization, blood vessels, and/or endothelial cells.

### Transplantation and Methods of Treatment

In some embodiments, the esophageal raft cultures or esophageal raft cell compositions described herein are transplanted or grafted into a host organism, for example, as a treatment or an experimental model. In some embodiments, the transplant is performed after culturing the raft culture for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days of culture within a range defined by any two of the aforementioned days, for example, 1 to 50 days, 10 to 40 days, 20 to 30 days, 1 to 30 days, or 20 to 50 days. In some embodiments, the raft culture is mature enough for transplantation and/or study a number of days before esophageal organoids prepared by other methods known in the art are at the same or similar mature state, wherein the number of days is, is about, is at least, is at least about, is not more than, or is not more than about, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any number of days within a range defined by any two of the aforementioned number of days, for example, 5 to 40 days, 10 to 40 days, 20 to 30 days, or 5 to 30 days. In some embodiments, the host organism is a mammal. In some embodiments, the host organism is an immunodeficient mammal. In some embodiments, the host organism is an immunodeficient mouse. In some embodiments, the host organism is a monkey, cat, dog, hamster, or rat. In some embodiments, the host organism is an immunocompromised monkey, cat, dog, hamster, or rat. In some embodiments, the host organism is a human. In some embodiments, the host organism is an immunodeficient human. In some embodiments, the host organism is an immunocompetent human. In some embodiments, the host organism is an immunocompetent human treated with immunosuppressants. In some embodiments, the raft culture is autologous to the host organism. In some embodiments, the raft culture is allogeneic to the host organism. In some embodiments, the host organism is a mammal that is in need of an esophageal transplant or graft. In some embodiments, the host organism is a human that is in need of an esophageal transplant or graft.

In some embodiments, the esophageal raft cultures or esophageal raft cell compositions serve as clinically beneficial tissue that can be used to study or treat a variety of different disease states, including but not limited to achalasia, Barrett's esophagus, esophageal cancer, gastroesophageal reflux disease (GERD), dysphagia, heartburn, eosinophilic esophagitis, paraoesophageal hernia, or esophageal perforation. In some embodiments, the esophageal raft cultures or esophageal raft cell compositions are used to assess pharmacological behavior, cell signaling, peristalsis, cancer formation and migration, or transplant/grafting, or any combination thereof.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the disclosure, as it is described herein and in the claims.

### Example 1. Generation of Esophageal Raft Cultures Comprising Epithelium and Mesenchyme

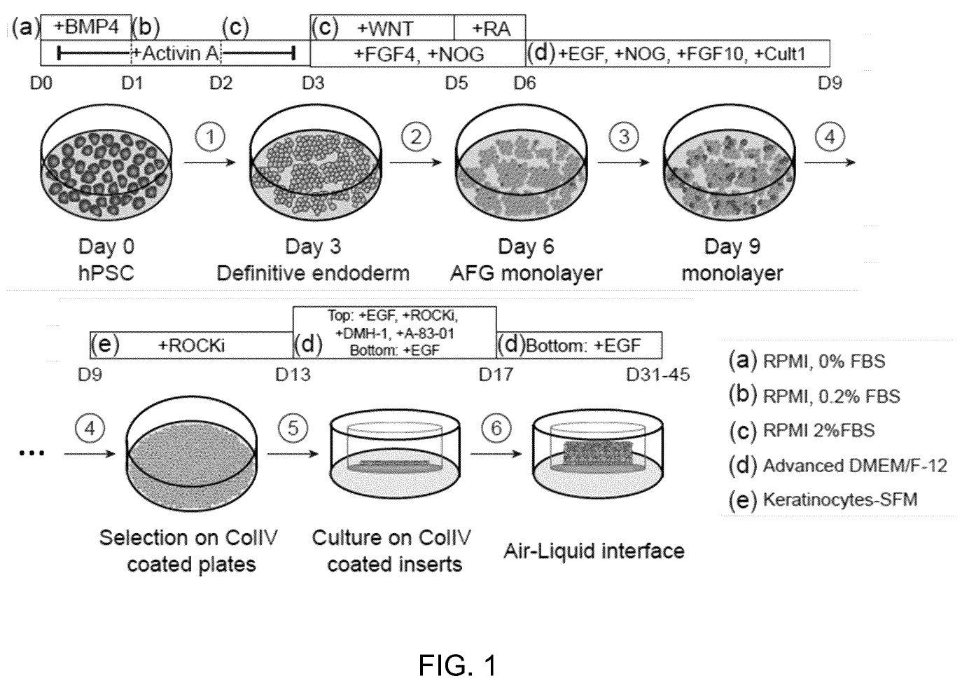

Esophageal raft cultures were produced according to the exemplary schematic depicted in FIG. 1.

Human PSCs (hPSCs) were cultured on hESC-qualified Matrigel-coated 10 cm plates and differentiated into esophageal monolayer (day 9). From day 0 to 6, growth medium with the provided supplements was changed daily. From day 0 to 1, hPSCs were cultured in RPMI 1640 supplemented with 50 ng/mL BMP4 (R&D Systems) and 100 ng/mL Activin A (R&D Systems). From day 1 to 2, hPSCs were cultured in RPMI 1640 supplemented with 100 ng/mL Activin A and 0.2% fetal bovine serum (FBS). From day 2 to 3, hPSCs were cultured in RPMI 1640 supplemented with 100 ng/mL Activin A and 2% FBS. At the end of day 3, hPSCs were differentiated into definitive endoderm cells.

Definitive endoderm cells were then differentiated into anterior foregut monolayer cells according to the following. From days 3 to 5, definitive endoderm cells were cultured in RPMI 1640 supplemented with 500 ng/mL Wnt3a (R&D systems), 500 ng/mL FGF4 (R&D Systems), 200 ng/mL Noggin (BMP inhibitor, R&D systems) and 2% FBS. From day 5 to 6, definitive endoderm cells were cultured in RPMI 1640 supplemented with 500 ng/mL FGF4, 200 ng/mL Noggin, 2 μM retinoic acid (RA, Sigma) and 2% FBS. At the end of day 6, definitive endoderm cells were differentiated into anterior foregut monolayer cells.

Anterior foregut monolayer cells were then differentiated into dorsal anterior foregut cells and esophageal progenitor cells. From days 6 to 9, anterior foregut monolayer cells were cultured in Advanced DMEM/F12 supplemented with 100 ng/mL EGF (R&D Systems), 200 ng/mL Noggin, and 50 ng/mL FGF10 to pattern to dorsal anterior foregut cells.

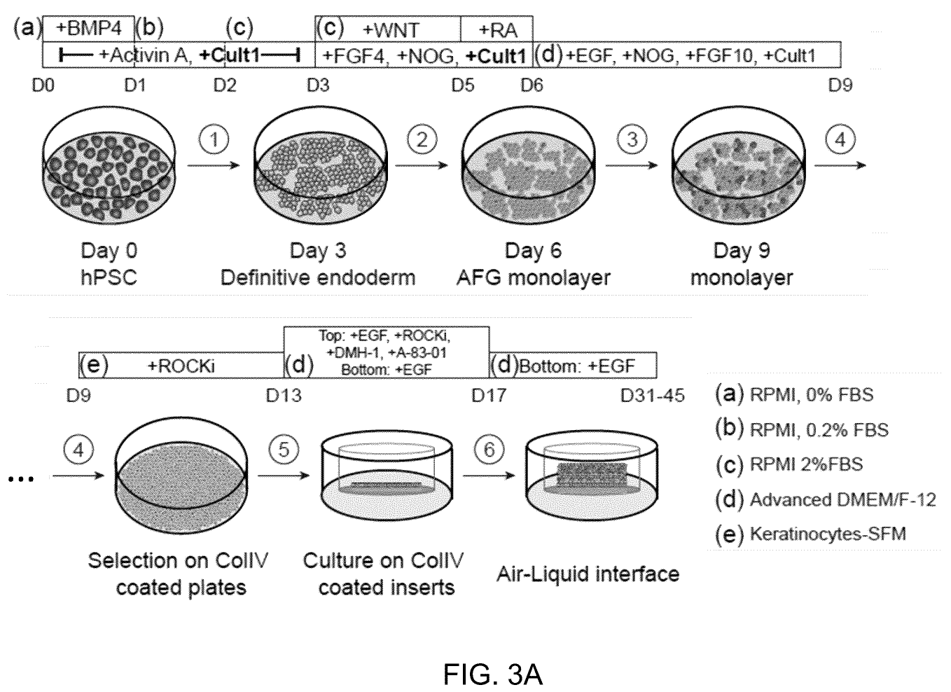

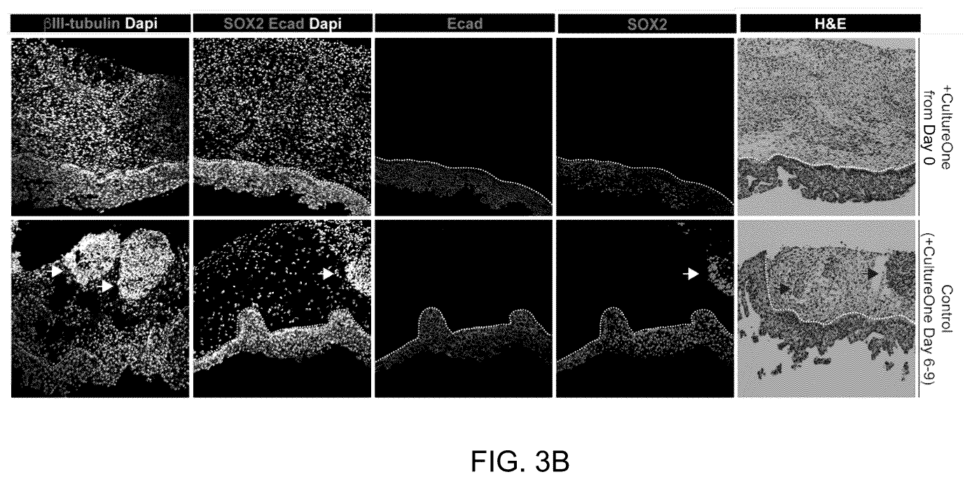

Optionally, lx CultureOne supplement (Cult1) (GIBCO) can be added from days 6-9 (as shown in FIG. 1), in addition to the other growth factors added at each day as provided herein. Alternatively, FIG. 3A shows 1× CultureOne optionally being added starting from day 0 until day 9. CultureOne was removed from culture from day 9 onward. CultureOne can be added to reduce neuronal contamination, and earlier addition further reduces neuronal contamination in the mesenchyme layer of the raft cultures. Alternative neuronal progenitor inhibitors are envisioned, such as cytarabine (ara-C). In other embodiments, CultureOne supplement is not included in the culture conditions.

On day 9, differentiated dorsal anterior foregut monolayer was dissociated to a single cell suspension using Accutase and cultured at approximately 1.8×10⁴ cells/cm² on collagen type IV (from human placenta)-coated plates (1.5 μg collagen/cm²) in Keratinocyte serum free medium (SFM) (GIBCO, Carlsbad, CA, USA) supplemented with 10 μM Y-27632 (ROCK inhibitor) until confluent (5 to 6 days) with growth medium changed every other day to differentiate to esophageal progenitor cells.

When the esophageal progenitor cells reach confluency, they were dissociated into a single cell suspension using 0.05% trypsin-EDTA and cultured on collagen type IV-coated (1.5 μg collagen/cm²) 3 μm pore size polycarbonate membrane cell inserts (Corning). For the first 5 days of culture on cell inserts, cells were cultured with fresh media daily in the top (insert) and bottom (plate) compartments. The top compartment was supplied with Advanced DMEM/F12 supplemented with 100 ng/mL EGF, 10 μM Y-27632, 1 μM DMH1 and 1 μM A 83-01 (SMAD inhibitors). The bottom compartment was supplied with Advanced DMEM/F12 supplemented with 100 ng/mL EGF. After 5 days, the cells were moved to an air-liquid interface and cultured with fresh media daily in the bottom compartment only. In this way, the epithelium of the raft culture, which is the more apical layer, is exposed to the air.

Cells at all stages were cultured at standard 37° C., 5% CO₂ incubation conditions.

### Example 2. Observations of Esophageal Raft Cultures

Esophageal raft cultures produced by the methods described herein were able to be differentiated and cultured in a 10 cm plate format rather than 24-well plate formats. There was no spheroid/organoid stage before differentiation into the raft cultures. Instead, cells were cultured in a monolayer and then in cell inserts. No feeder cell substrate (e.g. rat collagen type I matrix with irradiated mouse fibroblasts) was necessary for culturing the raft cultures. Instead, the plates and inserts, notably the surfaces to which the cells are contacted or the permeable surface of the inserts, were coated with human-derived type IV collagen. Matrigel and other basement membrane matrices were also not required. This has important considerations for scaling to larger cultures and cGMP production. The air-liquid interface started approximately two weeks after the start of esophageal differentiation. In comparison, previous esophageal raft culture and organoid protocols involved air-liquid interface initiation approximately 40 days after start of differentiation.

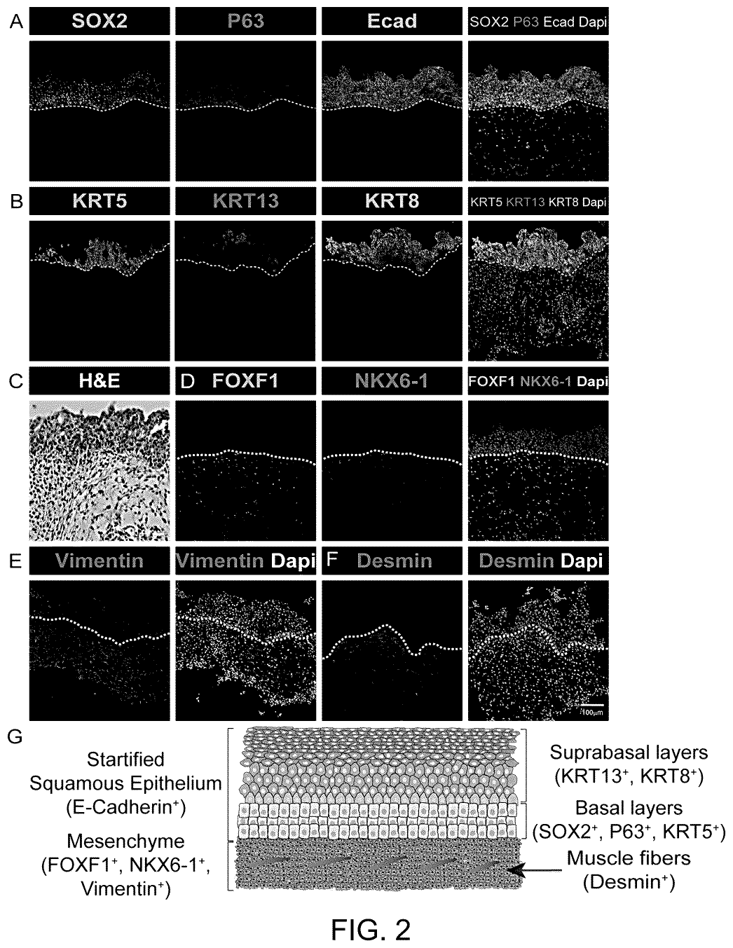

Importantly, the esophageal raft cultures described herein comprise both epithelium and mesenchyme, whereas previous raft cultures and organoids lack a mesenchyme. While there was a small population of mesenchyme progenitor cells in the day 6 to day 9 monolayer, the mesenchyme population expanded during the culture on Collagen type IV coated plates in supplemented Keratinocyte SFM medium, from day 9 to 14 (as described in Example 1). The use of the entire monolayer, instead of only collecting spontaneously emerging spheroids, as in previous protocols, allowed for the expansion of the small progenitor population that is otherwise lost. FIG. 2 demonstrates the morphology of the esophageal raft culture. The stratified squamous epithelium, marked by E-cadherin, is subdivided into a suprabasal layer, marked by Keratin 13 (KRT13) and Keratin 8 (KRT8), and a basal layer, marked by SOX2, P63, and Keratin 5 (KRT5). Under the epithelium, a mesenchyme, marked by mesenchymal cell markers FOXF1, NKX6-1, and Vimentin, contains differentiated myocytes (Desmin).

FIG. 3B depicts immunofluorescence images comparing esophageal raft cultures when CultureOne supplement was used either between days 0-9 or days 6-9 of culture. Presence of neuronal cell types, which might not be desirable, are indicated by expression of SOX2 (arrow) or βIII-tubulin (arrow) within the Ecad-negative mesenchymal layer. SOX2 is normally expressed in the Ecad+ esophageal epithelium.

### Example 3. Enteric Neural Crest Cells (ENCC) Differentiation and Co-Culture into Esophageal Raft Cultures

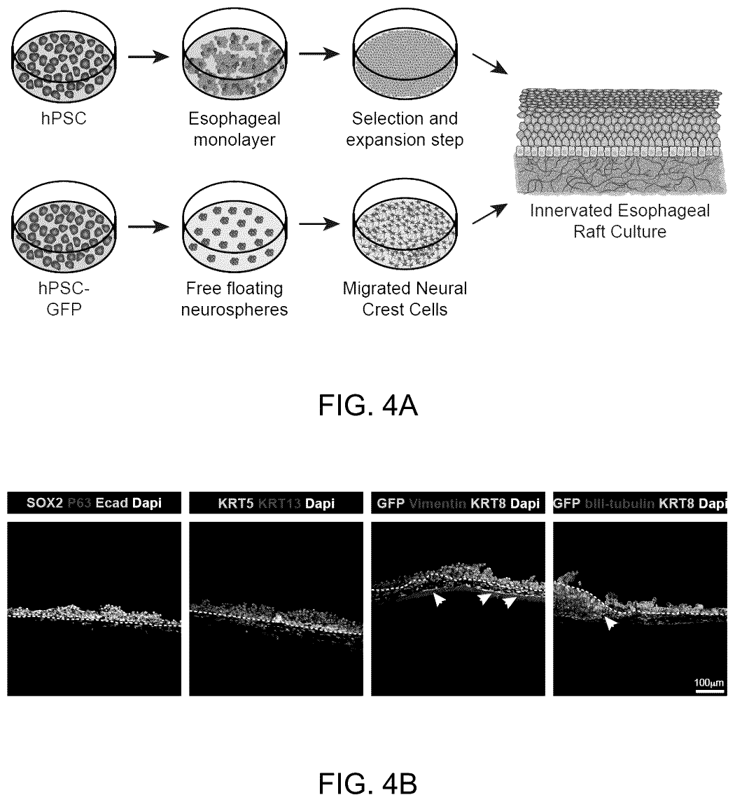

Esophageal raft cultures can be innervated by combination with enteric neural crest cells (ENCCs) during culturing and differentiation (FIG. 4A).

hPSCs were cultured on hESC-qualified Matrigel-coated plates and were treated with collagenase IV (500 U/mL, Gibco) in mTeSR1 for 60-90 minutes at 37° C. to detach colonies. Cells were then washed with DMEM/F-12 (Gibco) and transferred into a 15 mL conical tube. Once cells pelleted at the bottom of the tube, DMEM/F-12 was removed and cells were gently triturated and resuspended in neural induction media. Neural induction media is made of a 1:1 ratio of DMEM/F12-GlutaMAX (Gibco) and Neurobasal Medium (Gibco) supplemented with B27 supplement (0.5×, Gibco), N2 supplement (0.5×, Gibco), pen-strep (lx, Gibco), insulin (5 μg/mL, Sigma-Aldrich), FGF2 (20 ng/mL, R&D Systems) and EGF (20 ng/mL, R&D Systems). Cells were cultured on non-tissue culture-treated 60 mm petri dishes (Fisherbrand). Neural induction media was changed daily for 5 days, with 2 μM retinoic acid (RA) added to the media on day 4 and day 5 for posteriorizing. On day 6, free-floating neurospheres were collected and cultured on human fibronectin (HFN)-coated plates (3 μg/cm² diluted in PBS, Corning) in neural induction media (without RA) for an additional 4 days with daily medium changes. Confluent cells were then collected by a brief 2-3 minute Accutase treatment and were re-cultured on HFN-coated plates for an additional 4 days in neural induction media without RA. At this stage, cells were collected again by a brief Accutase treatment, counted, and re-combined with esophageal progenitor cells (day 13 of the esophageal raft culture differentiation process disclosed herein) and cultured on cell inserts. The ENCC-esophageal progenitor seeding ratio is approximately 2:1. The co-culture was maintained in the same conditions as esophageal cultures lacking ENCC: the top compartment is supplied with Advanced DMEM/F12 supplemented with 100 ng/mL EGF, 10 μM Y-27632, 1 μM DMH1 and 1 μM A 83-01 (SMAD inhibitors). The bottom compartment is supplied with Advanced DMEM/F12 supplemented with 100 ng/mL EGF. After 5 days, the cells are moved to an air-liquid interface and cultured with fresh media daily in the bottom compartment only.

FIG. 4B shows representative markers for the innervated esophageal raft cultures. The raft cultures express typical esophageal epithelial markers (SOX2, P63, KRT5, KRT13, and KRT8) and the general epithelium marker E-cadherin (Ecad). The incorporated GFP-expressing ENCCs, marked by arrows, innervate the esophageal raft culture mesenchyme, marked by expression of vimentin and the neuronal marker βIII-tubulin. βIII-tubulin expression is co-localized with GFP expression, indicating that the innervating neurons originated from hPSC-GFP that were directly differentiated into ENCC, and not from neural contamination during the esophageal raft differentiation.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

With respect to the use of “e.g.”, it is understood to mean “for example” and is therefore a non-limiting example.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference for any particular disclosure herein and in their entirety, and are hereby made a part of this specification. To the extent publications and patents or patent applications incorporated by reference contradict the disclosure contained in the specification, the specification is intended to supersede and/or take precedence over any such contradictory material.

What is claimed is:

1. An in vitro esophageal raft culture comprising:
a stratified squamous epithelium layer comprising a suprabasal layer and a basal layer; and
a mesenchyme layer comprising muscle fibers;
wherein the stratified squamous epithelium is E-cadherin⁺, the suprabasal layer is KRT13⁺ and KRT8⁺, and the basal layer is SOX2⁺, P63⁺, and KRT5⁺; and
wherein the mesenchyme layer is FOXF1⁺, NKX6-1⁺, and vimentin⁺, and the muscle fibers are desmin⁺.

2. The esophageal raft culture of any one of the preceding claims, wherein the esophageal raft culture lacks a lamina propria layer, or has a reduced lamina propria layer compared to esophageal tissue from an adult animal of the same species as the raft culture.

3. The esophageal raft culture of any one of the preceding claims, further comprising a growth medium, optionally DMEM/F12.

4. The esophageal raft culture of any one of the preceding claims,
wherein the esophageal raft culture is located in, and/or on a surface of, an insert member comprising a surface that is permeable to the growth medium but not cells, and the insert member is positioned within a tissue culture container; and
optionally wherein the esophageal raft culture is positioned on the surface that is permeable to the growth medium but not cells.

5. The esophageal raft culture of claim 4, wherein at least a portion of the insert member, optionally the surface that is permeable to the growth medium but not the cells, is coated with an extracellular matrix or a component thereof.

6. The esophageal raft culture of claim 5, wherein the extracellular matrix or a component thereof is derived from human.

7. The esophageal raft culture of claim 5 or 6, wherein the extracellular matrix or a component thereof comprises human collagen type IV.

8. The esophageal raft culture of any one of claims 5-7, wherein the extracellular matrix or a component thereof does not comprise rat collage type I matrix or Matrigel.

9. The esophageal raft culture of any one of claims 4-8, wherein the insert member and/or tissue culture container contain an amount of growth medium such that the esophageal raft culture is fully submerged in the growth medium.

10. The esophageal raft culture of claim 9, wherein the growth medium contained within the insert member further comprises an EGF pathway activator, a ROCK inhibitor, a SMAD inhibitor, or any combination thereof, and the growth medium contained within the tissue culture container comprises an EGF pathway activator.

11. The esophageal raft culture of any one of claims 4-8, wherein the tissue culture container and/or insert member contain an amount of growth medium such that the esophageal raft culture is only partially submerged in the growth medium,
wherein the stratified squamous epithelium is only partially submerged or not submerged in the growth medium and forms and/or is located at an air-liquid interface.

12. The esophageal raft culture of claim 11, wherein the growth medium contained within the tissue culture container comprises an EGF pathway activator.

13. The esophageal raft culture of any one of claims 4-12, wherein the surface that is permeable of the insert member comprises a pore size that is, is about, is at least, is at least about, is not more than, or is not more than about, 0.1, 0.5, 1, 2, 3, 4, 5, 6, 7, 8, 9, or 10 μm, or any pore size within a range defined by any two of the aforementioned sizes.

14. The esophageal raft culture of any one of claims 4-13, wherein the surface that is permeable of the insert member comprises a pore size of 3 μm.

15. The esophageal raft culture of any one of claims 1-14, wherein the esophageal raft culture is effectively free of neuronal progenitor cells and/or βIII-tubulin+ neuronal cells.

16. The esophageal raft culture of any one of claims 1-14, wherein the esophageal raft culture further comprises enteric neural crest cells (ENCCs), neuronal progenitor cells and/or βIII-tubulin+ neuronal cells, such that the esophageal raft culture is an innervated esophageal raft culture, optionally wherein the neuronal progenitor cells are SOX10⁺.

17. The esophageal raft culture of any one of claims 1-16, wherein the esophageal raft culture does not have vascularization, blood vessels, and/or endothelial cells.

18. An in vitro cell culture comprising:
a population of esophageal progenitor cells derived from dorsal anterior foregut cells which have been treated with an EGF pathway activator, a BMP pathway inhibitor, an FGF pathway activator, or any combination thereof.

19. The cell culture of claim 18, wherein the dorsal anterior foregut cells have also been treated with a neuronal progenitor inhibitor, optionally CultureOne supplement or cytarabine.

20. The cell culture of claim 18 or 19, further comprising a growth medium, optionally a serum free medium, optionally Keratinocyte SFM.

21. The cell culture of claim 20, wherein the growth medium comprises an EGF pathway activator or bovine pituitary extract (BPE), or both.

22. The cell culture of claim 21, wherein:
the EGF pathway activator is at a concentration of about 1, 2, 3, 4, 5, 6, 7, 8, 9, 10, 11, 12, 13, 14, 15, 16, 17, 18, 19, or 20 ng/mL, or any concentration within a range defined by any two of the aforementioned concentrations; or
the BPE is at a concentration of about 5, 10, 20, 30, 40, 50, 60, 70, 80, 90, or 100 μg/mL, or any concentration within a range defined by any two of the aforementioned concentrations, or both.

23. The cell culture of any one of claims 18-22, wherein the cell culture is located in, and/or on a surface of, a tissue culture container.

24. The cell culture of claim 23, wherein at least a portion of the tissue culture container is coated with an extracellular matrix or a component thereof, and said population of esophageal progenitor cells are on or in contact with said portion.

25. The cell culture of claim 24, wherein the extracellular matrix or a component thereof is derived from human.

26. The cell culture of claim 24 or 25, wherein the extracellular matrix or a component thereof comprises human collagen type IV.

27. The cell culture of any one of claims 24-26, wherein the extracellular matrix or a component thereof does not comprise rat collage type I matrix or Matrigel.

28. The cell culture of any one of claims 18-27, further comprising a ROCK inhibitor.

29. The cell culture of any one of claims 18-28, further comprising enteric neural crest cells.

30. An in vitro cell culture comprising:
a population of anterior foregut cells treated with an EGF pathway activator, a BMP pathway inhibitor, an FGF pathway activator, or any combination thereof.

31. The cell culture of claim 30, wherein the anterior foregut cells are further treated with a neuronal progenitor inhibitor, optionally CultureOne supplement or cytarabine.

32. The cell culture of claim 30 or 31, further comprising a growth medium, optionally RPMI, optionally with FBS, optionally 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% FBS, or any percentage of FBS within a range defined by any two of the aforementioned percentages.

33. The cell culture of any one of claims 30-32, wherein said cell culture is located in, and/or on a surface of a tissue culture container.

34. The esophageal raft culture of any one of claims 1-17, or the cell culture of any one of claims 18-33, wherein the esophageal raft culture or cell culture has been grown for at least 1, 2, 3, 4, 5, 6, 7, or 8 days.

35. The esophageal raft culture or the cell culture of claim 34, wherein the esophageal raft culture or the cell culture have been derived from human induced pluripotent stem cells.

36. The esophageal raft culture or the cell culture of claim 34 or 35, wherein the esophageal raft culture or the cell culture is not derived from a spheroid or organoid.

37. A method of producing an esophageal raft culture, comprising:
(a) contacting anterior foregut cells with an EGF pathway activator, a BMP pathway inhibitor, an FGF pathway activator, or any combination thereof to differentiate the anterior foregut cells into dorsal anterior foregut cells;
(b) dissociating the dorsal anterior foregut cells from step (a) into single cells;
(c) culturing the dorsal anterior foregut cells in a first tissue culture container to differentiate the dorsal anterior foregut cells to esophageal progenitor cells;
(d) dissociating the esophageal progenitor cells from step (c) into single cells;
(e) culturing the esophageal progenitor cells in, and/or on a surface of, an insert member,
wherein the insert member is positioned within a second tissue culture container,
wherein the insert member comprises a surface that is permeable to a growth medium but not cells; and
wherein the insert member and second tissue culture container each contain an amount of growth medium such that the esophageal progenitor cells are fully submerged in the growth medium; and
(f) culturing the esophageal progenitor cells in the insert member, wherein the second tissue culture container and/or insert member contains an amount of growth medium such that the esophageal progenitor cells are only partially submerged in the growth medium.

38. The method of claim 37, wherein the anterior foregut cells are further contacted with a neuronal progenitor inhibitor, optionally CultureOne supplement or cytarabine.

39. The method of claim 37 or 38, wherein the esophageal progenitor cells are dissociated using a dissociation enzyme, optionally trypsin, chymotrypsin, collagenase, elastase, or Accutase.

40. The method of any one of claims 37-39, wherein at least a portion of the first tissue culture container and/or the second tissue culture container are coated with an extracellular matrix or a component thereof.

41. The method of claim 40, wherein the extracellular matrix or a component thereof is derived from human.

42. The method of claim 40 or 41, wherein the extracellular matrix or a component thereof comprises human collagen type IV.

43. The method of any one of claims 40-42, wherein the extracellular matrix or a component thereof does not comprise rat collagen type I matrix or Matrigel.

44. The method of any one of claims 37-43, wherein the contacting step of (a) takes place over at least 1, 2, 3, 4, or 5 days.

45. The method of any one of claims 37-44, wherein the culturing step of (c) takes place over at least 1, 2, 3, 4, or 5 days.

46. The method of any one of claims 37-45, wherein the culturing step of (e) takes place over at least 2, 3, 4, 5, 6, 7, or 8 days.

47. The method of any one of claims 37-46, wherein the culturing step of (f) takes place over at least 10, 11, 12, 13, 14, 15, 16, 17, 18, 19, or 20 days.

48. The method of any one of claims 37-47, wherein the dorsal anterior foregut cells of step (c) are cultured with an EGF pathway activator, BPE, a ROCK inhibitor, or any combination thereof.

49. The method of any one of claims 37-48, wherein the esophageal progenitor cells of step (e) are cultured with an EGF pathway activator, a ROCK inhibitor, a SMAD inhibitor, or any combination thereof in the growth medium of the insert member and EGF in the growth medium of the second tissue culture container.

50. The method of any one of claims 37-49, wherein the esophageal progenitor cells of step (f) are cultured with an EGF pathway activator in the growth medium of the second tissue culture container.

51. The method of any one of claims 37-50, wherein the anterior foregut cells have been derived from human induced pluripotent stem cells.

52. The method of any one of claims 37-51, wherein the anterior foregut cells have been derived from definitive endoderm cells, wherein the definitive endoderm cells have been derived from human induced pluripotent stem cells.

53. The method of claim 52, wherein the definitive endoderm cells have been treated with Wnt3a, FGF4, Noggin, or RA, or any combination thereof, to differentiate the definitive endoderm cells to anterior foregut cells.

54. The method of claim 53, wherein the definitive endoderm cells have been further treated with a neuronal progenitor inhibitor, optionally CultureOne supplement or cytarabine.

55. The method of any one of claims 52-54, wherein the definitive endoderm cells have been treated for 1, 2, 3, 4, or 5 days.

56. The method of any one of claims 51-55, wherein the human induced pluripotent stem cells have been treated with BMP4 and/or Activin A to differentiate the human induced pluripotent stem cells to definitive endoderm cells.

57. The method of claim 56, wherein the human induced pluripotent stem cells have been further treated with a neuronal progenitor inhibitor, optionally CultureOne supplement or cytarabine.

58. The method of any one of claims 51-57, wherein the human induced pluripotent stem cells have been treated for 1, 2, 3, 4, or 5 days.

59. The method of any one of claims 37-58, further comprising:
contacting human induced pluripotent stem cells with BMP4 and/or Activin A to differentiate the human induced pluripotent stem cells to definitive endoderm cells; and
contacting the definitive endoderm cells with Wnt, FGF4, Noggin, or RA, or any combination thereof to differentiate the definitive endoderm cells to the anterior foregut cells of step (a).

60. The method of claim 59, wherein the human induced pluripotent stem cells and/or the definitive endoderm cells are further contacted with a neuronal progenitor inhibitor, optionally CultureOne supplement or cytarabine.

61. The method of claim 59 or 60, wherein the human induced pluripotent stem cells are contacted for 1, 2, 3, 4 or 5 days.

62. The method of any one of claims 59-61, wherein the definitive endoderm cells are contacted for 1, 2, 3, 4, or 5 days.

63. The method of any one of claims 37-62, wherein the esophageal raft culture is effectively free of neuronal progenitor cells and/or βIII-tubulin+ neuronal cells.

64. The method of any one of claims 37-63, further comprising combining the dissociated esophageal progenitor cells of step (d) with enteric neural crest cells (ENCCs) and culturing the combined esophageal progenitor cells and ENCCs according to steps (e) and (f) to produce an innervated esophageal raft culture.

65. The method of claim 64, wherein the innervated esophageal raft culture comprises enteric neural crest cells (ENCCs), neuronal progenitor cells and/or βIII-tubulin+ neuronal cells, optionally wherein the neuronal progenitor cells are SOX10+.

66. The method of any one of claims 37-65, wherein the esophageal raft culture does not comprise vascularization, blood vessels, and/or endothelial cells.

67. An in vitro cell composition comprising:
a population of dorsal anterior foregut cells derived from anterior foregut cells treated with an EGF pathway activator, a BMP pathway inhibitor, an FGF pathway activator, or any combination thereof.

68. An in vitro cell composition comprising:
a population of anterior foregut cells treated with an EGF pathway activator, a BMP pathway inhibitor, an FGF pathway activator, or any combination thereof.

69. The cell composition of claim 68, wherein the population of anterior foregut cells is further treated with a neuronal progenitor inhibitor, optionally CultureOne supplement or cytarabine.

70. An in vitro esophageal raft cell composition comprising:
a stratified squamous epithelium layer comprising a suprabasal layer and a basal layer; and
a mesenchyme layer comprising muscle fibers;
wherein the stratified squamous epithelium is E-cadherin⁺, the suprabasal layer is KRT13⁺ and KRT8⁺, and the basal layer is SOX2⁺, P63⁺, and KRT5⁺; and
wherein the mesenchyme layer is FOXF1⁺, NKX6-1⁺, and vimentin⁺, and the muscle fibers are desmin⁺.

71. The esophageal raft cell composition of claim 70, wherein the esophageal raft cell composition is effectively free of neuronal progenitor cells and/or βIII-tubulin+ neuronal cells.

72. The esophageal raft cell composition of claim 70, wherein the esophageal raft cell composition further comprises enteric neural crest cells (ENCCs), neuronal progenitor cells and/or βIII-tubulin+ neuronal cells, such that the esophageal raft culture is an innervated esophageal raft culture, optionally wherein the neuronal progenitor cells are SOX10+.

73. The esophageal raft cell composition of any one of claims 70-72, wherein the esophageal raft cell composition does not comprise vascularization, blood vessels, and/or endothelial cells.

74. The esophageal raft cell composition of any one of the preceding claims, wherein the esophageal raft cell composition has a thickness of about 1, 5, 10, 20, 30, 40, 50, 60, 70, 80, 90, 100, 200, 300, 400, or 500 μm, or any thickness within a range defined by any two of the aforementioned thicknesses.

75. The esophageal raft cell composition of any one of the preceding claims, wherein the esophageal raft cell composition has a thickness of about 150, 200, 250, 300, 350, 400, 450, or 500 μm, or any thickness within a range defined by any two of the aforementioned thicknesses.

76. The esophageal raft cell composition of any one of the preceding claims, wherein the esophageal raft composition has a surface area of about 0.1, 0.5, 1, 5, 10, 15, 20, 25, 30, 40, 50, 60, 70, 80, 90, or 100 cm², or any surface area within a range defined by any two of the aforementioned surface areas.

77. The esophageal raft cell composition of any one of the preceding claims, wherein the esophageal raft composition has a surface area of about 0.1, 0.5, 1, 1.5, or 2 cm², or any surface area within a range defined by any two of the aforementioned surface areas.

78. The esophageal raft cell composition of any one of the preceding claims, wherein the esophageal raft composition has a volume of about 10⁻⁵, 10⁻⁴, 10⁻³, 10⁻², 10⁻¹, 1, 5 or 10 cm³, or any volume within a range defined by any two of the aforementioned volumes.

79. The esophageal raft cell composition of any one of the preceding claims, wherein the stratified squamous epithelium layer has a thickness of about 1, 5, 10, 20, 30, 40, 50, 60, 70, 80, 90, 100, 200, 300, 400, or 500 μm, or any thickness within a range defined by any two of the aforementioned thicknesses.

80. The esophageal raft cell composition of any one of the preceding claims, wherein the stratified squamous epithelium layer has a thickness of about 50, 60, 70, 80, 90, 100, 110, 120, 130, 140, 150, 160, 170, 180, 190, 200, 210, 220, 230, 240, or 250 μm, or any thickness within a range defined by any two of the aforementioned thicknesses.

81. The esophageal raft cell composition of any one of the preceding claims, wherein the suprabasal layer has a thickness of about 1, 5, 10, 20, 30, 40, 50, 60, 70, 80, 90, 100, 200, 300, 400, or 500 μm, or any thickness within a range defined by any two of the aforementioned thicknesses.

82. The esophageal raft cell composition of any one of the preceding claims, wherein the suprabasal layer has a thickness of about 80, 90, 100, 110, 120, 130, 140, 150, 160, 170, 180, 190, or 200 μm, or any thickness within a range defined by any two of the aforementioned thicknesses.

83. The esophageal raft cell composition of any one of the preceding claims, wherein the basal layer has a thickness of about 1, 5, 10, 20, 30, 40, 50, 60, 70, 80, 90, 100, 200, 300, 400, or 500 μm, or any thickness within a range defined by any two of the aforementioned thicknesses.

84. The esophageal raft cell composition of any one of the preceding claims, wherein the basal layer has a thickness of about 10, 20, 30, 40, 50, 60, 70, 80, 90, or 100 μm, or any thickness within a range defined by any two of the aforementioned thicknesses.

85. The esophageal raft cell composition of any one of the preceding claims, wherein the mesenchyme layer has a thickness of about 1, 5, 10, 20, 30, 40, 50, 60, 70, 80, 90, 100, 200, 300, 400, or 500 μm, or any thickness within a range defined by any two of the aforementioned thicknesses.

86. The esophageal raft cell composition of any one of the preceding claims, wherein the mesenchyme layer has a thickness of about 100, 150, 200, 250, 300, 350, or 400 μm, or any thickness within a range defined by any two of the aforementioned thicknesses.

87. The esophageal raft culture, cell culture, method, or esophageal raft cell composition of any one of the preceding claims, wherein the EGF pathway activator comprises EGF, TGF-alpha, AR, BTC, HB-EGF, EPR, tomoregulin, NRG-1, NRG-2, NRG-3, or NRG-4, or any combination thereof.

88. The esophageal raft culture, cell culture, method, or esophageal raft cell composition of any one of the preceding claims, wherein the EGF pathway activator is EGF.

89. The esophageal raft culture, cell culture, method, or esophageal raft cell composition of any one of the preceding claims, wherein the EGF pathway activator is provided at a concentration of about 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations.

90. The esophageal raft culture, cell culture, method, or esophageal raft cell composition of any one of the preceding claims, wherein the EGF pathway activator is provided at a concentration of 100 ng/mL or about 100 ng/mL.

91. The esophageal raft culture, cell culture, method, or esophageal raft cell composition of any one of the preceding claims, wherein the BMP pathway inhibitor comprises Noggin, RepSox, LY364947, LDN193189, SB431542, or any combination thereof.

92. The esophageal raft culture, cell culture, method, or esophageal raft cell composition of any one of the preceding claims, wherein the BMP pathway inhibitor is Noggin.

93. The esophageal raft culture, cell culture, method, or esophageal raft cell composition of any one of the preceding claims, wherein the BMP pathway inhibitor is provided at a concentration of about 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations.

94. The esophageal raft culture, cell culture, method, or esophageal raft cell composition of any one of the preceding claims, wherein the BMP pathway inhibitor is provided at a concentration of 200 ng/mL or about 200 ng/mL.

95. The esophageal raft culture, cell culture, method, or esophageal raft cell composition of any one of the preceding claims, wherein the FGF pathway activator comprises FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, or FGF23, or any combination thereof.

96. The esophageal raft culture, cell culture, method, or esophageal raft cell composition of any one of the preceding claims, wherein the FGF pathway activator is FGF10.

97. The esophageal raft culture, cell culture, method, or esophageal raft cell composition of any one of the preceding claims, wherein the FGF pathway activator is provided at a concentration of about 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations.

98. The esophageal raft culture, cell culture, method, or esophageal raft cell composition of any one of the preceding claims, wherein the FGF pathway activator is provided at a concentration of 50 ng/mL or about 50 ng/mL.

99. The esophageal raft culture, cell culture, method, or esophageal raft cell composition of any one of the preceding claims, wherein the ROCK inhibitor comprises Y-27632, Y-30141, Y-39983, Ki-23095, SLx-2119, thiazovivin, azaindole 1, fasudil, ripasudil, netarsudil, RKI-1447, or GSK429286A, or any combination thereof.

100. The esophageal raft culture, cell culture, method, or esophageal raft cell composition of any one of the preceding claims, wherein the ROCK inhibitor is Y-27632.

101. The esophageal raft culture, cell culture, method, or esophageal raft cell composition of any one of the preceding claims, wherein the ROCK inhibitor is provided at a concentration of about 1, 2, 3, 4, 5, 6, 7, 8, 9, 10, 11, 12, 13, 14, 15, 16, 17, 18, 19, or 20 μM, or any concentration within a range defined by any two of the aforementioned concentrations.

102. The esophageal raft culture, cell culture, method, or esophageal raft cell composition of any one of the preceding claims, wherein the ROCK inhibitor is provided at a concentration of 10 μM or about 10 μM.

103. The esophageal raft culture, cell culture, method, or esophageal raft cell composition of any one of the preceding claims, wherein the SMAD inhibitor comprises A-83-01, RepSox, LY365947, LY2109761, LY364947, SB431542, SB525334, SB505125, galunisertib, GW788388, LDN-193189, LDN-212854, hesperetin, or any combination thereof.

104. The esophageal raft culture, cell culture, method, or esophageal raft cell composition of any one of the preceding claims, wherein the SMAD inhibitor is DMH1 and A-83-01.

105. The esophageal raft culture, cell culture, method, or esophageal raft cell composition of any one of the preceding claims, wherein the SMAD inhibitor is provided at a concentration of about 0.1, 0.5, 1, 2, 3, 4, 5, 6, 7, 8, 9, or 10 μM, or any concentration within a range defined by any two of the aforementioned concentrations.

106. The esophageal raft culture, cell culture, method, or esophageal raft cell composition of any one of the preceding claims, wherein the SMAD inhibitor is provided at a concentration of 1 μM or about 1 μM.
